(19) United States
(12) Patent Application Publication
(10) Pub. No.: US 2018/0368966 A1
(43) Pub. Date: Dec. 27, 2018

(54) Artificial Graft Devices and Related Systems and Methods

(71) Applicant: Neograft Technologies, Inc., Taunton, MA (US)

(72) Inventor: Lorenzo SOLETTI, Pittsburgh, PA (US)

(21) Appl. No.: 15/977,894

(22) Filed: May 11, 2018

Related U.S. Application Data
(63) Continuation of application No. 15/108,276, filed on Jun. 24, 2016, now abandoned.
(60) Provisional application No. 61/921,196, filed on Dec. 27, 2013.

(51) Int. Cl. A61F 2/06; A61L 27/28; A61L 27/18; A61L 27/50; A61L 27/54; A61L 27/56; A61L 27/58

(52) U.S. Cl. CPC A61F 2/06; A61L 2430/36; A61F 2/064; A61L 27/18; A61F 2/062; A61L 27/50; A61L 27/54; A61L 27/56; A61L 27/58; A61F 2250/003; A61F 2250/0067; A61F 2210/0004; A61F 2250/0019; A61L 2300/42; A61L 2420/08; A61L 27/28

(57) ABSTRACT

In some aspects, a graft device can include a biodegradable inner layer, an outer layer, a first end portion, a second end portion, and a lumen therebetween. The biodegradable inner layer typically includes an inner surface and an outer surface. The outer layer typically includes a fiber matrix surrounding the outer surface of the inner layer. The graft device can include a reinforced end portion. At least 10% or at least 50% of the graft device can remain after 90 days of implantation. In some cases, at least 10% or at least 50% of the graft device can remain after 180 days of implantation. The graft device can include a kink-resisting element. The graft device can include at least one layer with a dynamic compliance less than or equal to at least one of: 20%/100 mmHg or 5%/100 mmHg.

### RELATED APPLICATIONS

This application claims the benefit of U.S. Provisional Application No. 61/921,196, filed Dec. 27, 2013, the contents of which are hereby incorporated herein by reference in their entirety.

This application is related to U.S. patent application Ser. No. 12/022,430, filed Jan. 30, 2008; U.S. patent application Ser. No. 13/515,996, filed Jun. 14, 2012; U.S. patent application Ser. No. 13/811,206, filed Jan. 18, 2013; U.S. patent application Ser. No. 13/979,243, filed Jul. 11, 2013; U.S. patent application Ser. No. 13/984,249, filed Aug. 7, 2013; U.S. patent application Ser. No. 14/354,025, filed Apr. 24, 2014; U.S. patent application Ser. No. 14/378,263, filed Aug. 12, 2014; International Patent Application Serial Number PCT/US2014/056371, filed Sep. 18, 2014; and International Patent Application Serial Number PCT/US2014/065839, filed Nov. 14, 2014; the contents of each of which are incorporated herein by reference in their entirety.

### TECHNICAL FIELD

This application relates generally to graft devices, and more particularly to graft devices for providing cardiovascular bypass for mammalian patients.

### BACKGROUND

Coronary artery disease, leading to myocardial infarction and ischemia, is currently a leading cause of morbidity and mortality worldwide. Current treatment alternatives consist of percutaneous transluminal angioplasty, stenting, and coronary artery bypass grafting (CABG). CABG can be carried out using either arterial or venous conduits and is the most effective and most widely used treatment to combat coronary arterial stenosis, with nearly 500,000 procedures being performed annually. In addition, there are approximately 80,000 lower extremity bypass surgeries performed annually. The venous conduit used for bypass procedures is most frequently the autogenous saphenous vein and remains the graft of choice for 95% of surgeons performing these bypass procedures. According to the American Heart Association, in 2004 there were 427,000 bypass procedures performed in 249,000 patients. The long term outcome of these procedures is limited due to occlusion of the graft vein or anastomotic site as a result of intimal hyperplasia (IH), which can occur over a timeframe of months to years.

Development of successful small diameter synthetic or tissue engineered vascular grafts has yet to be accomplished and use of arterial grafts (internal mammary, radial, or gastroepiploic arteries, for example) is limited by the short size, small diameter and availability of these veins. Despite their wide use, failure of arterial vein grafts (AVGs) remains a major problem: 12% to 27% of AVGs become occluded in the first year with a subsequent annual occlusive rate of 2% to 4%. Patients with failed AVGs usually require clinical intervention such as an additional surgery.

IH accounts for 20% to 40% of all AVG failures within the first 5 years after CABG surgery. Several studies have determined that IH develops, to some extent, in all mature AVGs and this development is regarded by many as an unavoidable response of the vein to grafting. III is characterized by phenotypic modulation, followed by de-adhesion and migration of medial and adventitial smooth muscle cells (SMCs) and myofibroblasts into the intima where they proliferate.

### SUMMARY

For these and other reasons, there is a need for systems, methods and devices that provide enhanced graft devices for mammalian patients. Desirably, the systems, methods, and devices will improve long term patency and reduce (e.g. minimize) surgical and device complications such as those caused by kinking of graft devices, or those caused by insufficient durability of the graft leading to aneurysm formation.

Embodiments of the present inventive concepts can be directed to graft devices for mammalian patients, as well as systems and methods for producing these graft devices.

In some aspects, a graft device comprises a biodegradable inner layer, an outer layer, a first end portion, a second end portion and a lumen therebetween. The biodegradable inner layer can include an inner surface and an outer surface. The outer layer comprises a fiber matrix and surrounds the outer surface of the inner layer.

In some embodiments, the graft device further comprises at least one of: a reinforced first end portion or a reinforced second end portion. In some embodiments, at least 10% of the graft device remains after 90 days of implantation. In some embodiments, at least 50% of the graft device remains after 90 days of implantation. In some embodiments, at least 10% of the graft device remains after 180 days of implantation. In some embodiments, at least 50% of the graft device remains after 180 days of implantation.

In some embodiments, the graft device further comprises a kink-resisting element.

In some embodiments, the graft device comprises at least one layer with a dynamic compliance less than or equal to at least one of: 20%/100 mmHg or 5%/100 mmHg.

In some embodiments, the graft device comprises a coronary arterial graft. In some embodiments, the graft device comprises a peripheral arterial graft. In some embodiments, the graft device is constructed and arranged to produce at least one of: a neo-artery or a neo-vein.

In some embodiments, the graft device comprises 3 or more layers. The 3 or more layers can comprise an inner layer constructed and arranged to biodegrade faster than an outer layer. The 3 or more layers can comprise a middle layer and two surrounding layers, and the middle layer can be constructed and arranged to biodegrade faster than the two surrounding layers.

In some embodiments, the graft device comprises a compliance less that or equal to 20%/100 mmHg. The lumen can comprise a diameter between 2.0 mm and 5.0 mm.

In some embodiments, the inner layer comprises biodegradable polyester. The biodegradable polyester can comprise poly(glycerol sebacate) (PGS). In some embodiments, the inner layer comprises a polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations of one or more of these or other materials.

In some embodiments, the inner layer comprises a first material and a second material. The first material can comprise a first hardness and the second material can comprise a second, different hardness. The first material hardness can be less than the second material hardness, and the first material can comprise segments including polydimethylsiloxane and polyhexamethylene oxide, and the second material can comprise segments including aromatic methylene diphenyl isocyanate. The first material and the second material can be constructed and arranged to biodegrade at different rates. The first material and the second material can comprise different molecular weights. The first material and the second material can comprise different degrees of cross-linking.

In some embodiments, the inner layer comprises a polymer selected from the group consisting of: polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations of one or more of these or other materials.

In some embodiments, the inner layer comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a biodegradable polyurethane; a polyphosphazene; and combinations of one or more of these or other materials.

In some embodiments, the inner layer further comprises an agent constructed and arranged to be released over time. In some embodiments, the inner layer further comprises a non-biodegradable material. In some embodiments, the inner layer is constructed and arranged to biodegrade primarily via surface erosion.

In some embodiments, the inner layer comprises at least a portion with minimal porosity. The minimal porosity portion can comprise an outermost portion of the inner layer. The minimal porosity portion can comprise a full circumferential sub-layer of the inner layer. The inner layer can comprise a thickness less than or equal to 600 μm and the minimal porosity portion can comprise a thickness less than or equal to 510 μm. The minimal porosity portion can comprise a compliance chamber.

In some embodiments, the inner layer comprises a relatively uniform outer diameter along its length. In some embodiments, the inner layer comprises a variable outer diameter along its length. In some embodiments, the inner layer comprises a relatively straight geometry. In some embodiments, the inner layer comprises a curved geometry.

In some embodiments, the inner layer further comprises one or more of: microspheres; nanoparticles such as polymer-layer silicates; metal; metal alloy; ceramic; glass; a self-assembled monolayer; and a biomimetic material such as a phospholipids layer with inherent anti-thrombogenic properties.

In some embodiments, the inner layer comprises a construction selected from the group consisting of: homogenous construction; heterogeneous construction; crystalline construction; semi-crystalline construction; amorphous construction; fibrous construction; open-celled construction; closed celled construction; woven construction; interconnected pore construction such as that produced by spherical aggregation, spherical particle-leaching such as salt-leaching, thermally-induced phase separation, and/or thermally-induced particle-leaching; and combinations of one or more of these or other materials.

In some embodiments, the inner layer comprises at least a permeable portion. The permeable portion can be permeable to a material selected from the group consisting of: oxygen; a cellular nutrient; cells; water; blood and combinations of one or more of these materials.

In some embodiments, the graft further comprises a compliance chamber. The compliance chamber can be positioned at least one of: in, on or within the inner layer. The compliance chamber can comprise a relatively full circumferential sub-layer of the inner layer. The compliance chamber can comprise minimally porous material. The compliance chamber can comprise a foam construction. The outer layer can surround the compliance chamber.

In some embodiments, the inner layer comprises multiple sub-layers.

In some embodiments, the inner layer comprises a layer produced using a process selected from the group consisting of: particle-leaching (e.g. salt, wax and/or sugar particle leaching) following controlled dipping of a cylindrical rod into a bath of a solution containing undissolved particles of controlled size followed by dissolution of the particles to leave interconnected pores (e.g. via a freeze-drying step); particle-leaching (e.g. salt, wax and/or sugar particle leaching) following casting into a tubular mold of a solution containing undissolved particles of controlled size followed by dissolution of the particles to leave interconnected pores (e.g. via a freeze-drying step); thermally-induced separation of a solution following casting into a tubular mold followed by freeze-drying; freeze-drying of synthetic and/or biological-based hydrogels cast into a tubular mold or dipped in a bath; freeze-drying of flat sheets of de-cellularized tissues rolled onto a cylindrical template; freeze-drying of de-cellularized tubular tissues; rolling of flat sheets of synthetic meshes of material around a cylindrical template; thermoplastic extrusion of tubular constructs followed by laser excimer micro and/or macro porosity creation to form a tubular mesh structure or a porous tubular structure; sintering of thermoplastic polymer particles; wire-network molding; synthesis of a polymer with high internal phase emulsions; and combinations of one or more of these processes.

In some embodiments, the inner layer comprises a layer produced using a device selected from the group consisting of: electrospinning device; melt-spinning device; melt-electrospinning device; 3D printer; micro-3D printer fused deposition modeling device; selective laser sintering device; laser excimer microdrilling device; sprayer; weaver; braider; knitter; dipping machine; casting machine; and combinations of one or more of these devices.

In some embodiments, the graft device further comprises a thromboresistant agent. The thromboresistant agent can be positioned about the inner surface of the inner layer. The thromboresistant agent can comprise heparin.

In some embodiments, the device comprises a device thickness and the lumen comprises a lumen diameter, and the device thickness can be related to the lumen diameter. The device thickness can be proportional to the lumen diameter.

In some embodiments, the device comprises a device thickness and the inner layer comprises an inner layer thickness, and the inner layer thickness can be greater than one-third the device thickness. In some embodiments, the device comprises a device thickness and the inner layer comprises an inner layer thickness, and the inner layer thickness can be less than one-half the device thickness.

In some embodiments, the device comprises a device thickness, and the device thickness comprises a thickness at least one of: more than or equal to 300 μm or less than or equal to 800 μm.

In some embodiments, the inner layer comprises an inner layer thickness, and the inner layer thickness comprises a thickness at least one of: more than or equal to 100 μm or less than or equal to 300 μm.

In some embodiments, the outer layer comprises an outer layer thickness, and the outer layer thickness comprises a thickness at least one of: more than or equal to 200 μm or less than or equal to 500 μm.

In some embodiments, the lumen comprises a diameter between 2.0 mm and 10.0 mm. The lumen can comprise a diameter between 2.0 mm and 5.0 mm.

In some embodiments, the fiber matrix is biodegradable. The fiber matrix can be constructed and arranged to biodegrade at a slower rate than the inner layer. In some embodiments, the fiber matrix comprises non-biodegradable materials. In some embodiments, the fiber matrix comprises both biodegradable and non-biodegradable materials. In some embodiments, the fiber matrix comprises poly(caprolactone) (PCL).

In some embodiments, the outer layer comprises multiple sub-layers. In some embodiments, the outer layer is constructed and arranged to limit compliance of the inner layer.

In some embodiments, the fiber matrix comprises a polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations of one or more of these materials.

In some embodiments, the fiber matrix comprises a first material and a second material. The first material can comprise a first hardness and the second material can comprise a second, different hardness. The first material hardness can be less than the second material hardness, and the first material can comprise segments including polydimethylsiloxane and polyhexamethylene oxide, and the second material can comprise segments including aromatic methylene diphenyl isocyanate.

In some embodiments, the inner layer comprises a polymer selected from the group consisting of: polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations of one or more of these materials.

In some embodiments, the inner layer comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a biodegradable polyurethane; a polyphosphazene; and combinations of one or more of these materials.

In some embodiments, the inner layer further comprises an agent constructed and arranged to be released over time.

In some embodiments, the graft further comprises pores. The pores can be positioned within the inner layer. The inner layer can comprise a first sub-layer and a second sub-layer. The pores can comprise a first set of pores within the first sub-layer and a second set of pores with the second sub-layer, and the first set of pores can comprise a different average diameter than the second set of pores. The second sub-layer can comprise minimal porosity. The second layer can comprise a compliance chamber. The second layer can circumferentially surround the first layer. The pores can be positioned in the outer layer. The pores can comprise diameters ranging from 10 μm to 100 μm. The pores can comprise diameters ranging from 20 μm to 30 μm. The pores can be positioned in a partial circumferential portion of the inner layer. The pores can be positioned in a full circumferential portion of the inner layer. The pores can be positioned in an innermost sub-layer of the inner layer. The pores can comprise a first set of pores and a second set of pores. The first set of pores can comprise a different average diameter than the second set of pores. The inner layer can comprise a first sub-layer comprising the first set of pores and a second sub-layer comprising the second set of pores. The pores can comprise interconnecting pores. At least 50% of the pores can comprise interconnecting pores. The interconnectivity can vary along a radial direction of the graft device. The interconnectivity can vary at least one of: continuously or discretely. The pores can comprise a first set of pores proximate the inner layer inner surface, and a second set of pores proximate the inner layer outer surface, and the first set of pores can comprise a higher interconnectivity than the second set of pores.

In some embodiments, at least one of the end portions comprises a reinforced end portion constructed and arranged to support an anastomotic connection. The first end portion can comprise a first reinforced end portion and the second end portion can comprise a second reinforced end portion. The reinforced end portion can comprise a bundle of small fibers. The reinforced end portion can comprise a tear-resistant coating. The reinforcing end portion can comprise a reinforcing element. The reinforcing element can comprise a full circumferential reinforcing element. The reinforcing element can comprise a reinforcing band. The reinforcing band can comprise a fabric band. The reinforcing element can comprise an anastomotic clip. The reinforcing end portion can comprise a thickened portion of at least one of: the inner layer or the outer layer.

In some embodiments, the graft further comprises a kink-resisting element. The kink-resisting element can comprise multiple kink-resisting elements. The kink-resisting element can be positioned between the inner layer and the outer layer. The outer layer can comprise a first sub-layer and a second sub-layer, and the kink-resisting element can he positioned between the first sub-layer and the second sub-layer. The kink-resisting element can comprise a spine. The spine can comprise multiple interdigitating projections. The kink-resisting element can comprise multiple rings. The kink-resisting element can comprise a biodegradable material. The kink-resisting element biodegradable material can be constructed and arranged to biodegrade slower than the inner layer. The inner layer can comprise a first material and the kink-resisting element can comprise a second material similar to the first material. The outer layer can comprise a first material and the kink-resisting element can comprise a second material similar to the first material. The kink-resisting element can comprise a metal. The kink-resisting element can comprise a biodegradable metal. The kink-resisting element can be constructed and arranged to avoid a significant change in a mechanical property of the device proximate the kink-resisting element. The kink-resisting element can comprise free ended strands of material. The kink-resisting element can comprise particles. The particles can be constructed and arranged to allow suture to pass therethrough. The kink-resisting clement can be constructed and arranged to provide one or more functions selected from the group consisting of: minimizing undesirable conditions, such as buckling, kinking, inner layer deformation, luminal deformation, stasis, flows characterized by significant secondary components of velocity vectors such as vortical, recirculating or turbulent flows, luminal collapse, and/or thrombus formation; preserving laminar flow such as preserving laminar flow with minimal secondary components of velocity, such as blood flow through the graft device, blood flow proximal to the graft device and/or blood flow distal to the graft device; preventing bending and/or allowing proper bending of the graft device, such as bending that occurs during and/or after the implantation procedure; preventing accumulation of debris; preventing stress concentration on the tubular wall; maintaining a defined geometry in the inner layer; preventing axial rotation about the length of the inner layer; and combinations thereof. The outer layer can comprise a first elastic moduli and the kink-resisting element can comprise a second elastic moduli similar to the first elastic moduli. The kink-resisting element can comprise a resiliently biased element.

In some embodiments, the graft device further comprises a coating. The coating can comprise a thromboresistant coating. The thromboresistant coating can comprise heparin. The thromboresistant coating can comprise a coating positioned on the inner surface of the inner layer. The coating can comprise an adhesive. The adhesive coating can comprise a coating positioned on the outer surface of the inner layer. The coating can comprise harvested tissue. The coating can comprise endothelial cells. The harvested tissue coating is positioned on the inner surface of the inner layer. The coating can be constructed and arranged to provide a function selected from the group consisting of: anti-thrombogenicity; anti-proliferation; anti-calcification; vasorelaxation; and combinations of one or more of these functions.

In some embodiments, the graft device further comprises at least a third end portion. The first end portion can comprise a first diameter, the second end portion can comprise a second diameter and the third end portion can comprise a third diameter, and the first diameter can be larger than at least one of: the second diameter or the third diameter.

According to another aspect, this disclosure relates to methods for producing graft devices as disclosed herein.

In some embodiments, the inner layer is created using a particle leaching process.

In some embodiments, the outer layer is created using a fiber matrix delivery device, such as an electrospinning device.

In some embodiments, the method comprises reinforcing at least one end portion of the device.

According to another aspect, this disclosure relates to systems for producing the graft devices disclosed herein.

In some embodiments, the systems comprise a fiber matrix delivery assembly, such as an electrospinning unit. In some embodiments, the system comprises a polymer solution.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a side, partial cutaway view of an example graft device with an inner layer and a fiber matrix outer layer.

FIG. 1A is an end, partial cutaway view of the graft device of FIG. 1.

FIG. 2 is a side view of an example graft device including a bifurcation.

FIG. 3A is a sectional view of an example embodiment of the graft device of FIG. 1, having a tubular conduit and a surrounding fiber matrix.

FIG. 3B is a sectional view of another example embodiment of the graft device of FIG. 1, having a tubular conduit, a spine, and a surrounding fiber matrix.

FIG. 4 is a schematic view of an example system for producing graft devices with an inner layer and an electrospun fiber matrix outer layer.

### DETAILED DESCRIPTION

The terminology used herein is for the purpose of describing particular example embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

Provided herein are graft devices for implantation in a mammalian patient, such as to temporarily or chronically carry fluids such as blood or other body fluids from a first anatomical location to a second anatomical location, such as from the aorta to a cardiac artery. Following implantation of the graft device, a new blood vessel (e.g. a neo-artery or neo-vein) can be progressively formed by remodeling mechanisms such as cellular infiltration, proliferation, fusion/differentiation and integration, followed by matrix synthesis and rearrangement. These mechanisms can create a new structure that ultimately replaces a portion or the whole existing structure of the graft device, such as to support the flow of blood. In some embodiments, the graft devices described herein support host tissue remodeling exclusively via a host-mediated regenerative processes, without the preliminary inclusion of cells or other biological factors (such as growth factors or other proteins) prior to implantation. In these embodiments, the graft device may not have been subjected to any mechanical preconditioning. The resulting remodeled neo-artery can include a confluent endothelium and structural smooth muscle layers, which can be contractile and responsive to autonomic signals. The neo-artery can also include protein components such as multi-structural elastin and collagen fibers, proteoglycans and glycosaminoglycan, and can exhibit resilient and compliant mechanical properties sufficient to support arterial flow of blood for long periods of time. In some embodiments, the graft device is replaced by a standard inflammatory response leading to the formation of a mature fibrous collagenous capsule comprising mainly fibroblastic and myofibroblastic cellular components (with minimal presence of the other aforementioned structural proteins).

The graft devices of the present inventive concepts can include an inner layer and an outer layer. The inner layer and/or the outer layer can each comprise one or more sub-layers (hereinafter simply “layers”). The inner layer can be bioabsorbable, biodegradable, bioerodible and/or otherwise lose structural integrity over time with or without chemical degradation (hereinafter “biodegradable”) and the outer layer can comprise a fiber matrix applied about an outer surface of the inner layer. The outer layer can comprise one or more layers, such as one or more biodegradable or non-biodegradable layers, such as an outer layer comprising at least one biodegradable layer and/or at least one non-biodegradable layer. The fiber matrix can be applied with one or more of: an electrospinning device; a melt-spinning device; a melt-electrospinning device; a misting assembly; a sprayer; an electrosprayer; a fuse deposition device; a selective laser sintering device; a three-dimensional printer; or other fiber matrix delivery device. The graft devices can comprise a coronary arterial graft and/or a peripheral arterial graft (i.e. be constructed and arranged to provide blood to a coronary artery and/or a peripheral artery of the patient). In a clinical procedure, end-to-side anastomotic connections are typically used to attach the graft device to a source of oxygenated blood and a diseased artery (e.g. between the aorta and a diseased coronary artery in a cardiovascular bypass procedure). Alternatively, a side-to-side anastomosis can be used, such as to attach an end of the graft device to multiple arteries in a serial fashion.

The graft devices described herein can include one or more features constructed and arranged to perform a function selected from the group consisting of: limit compliance to withstand arterial pressures and maintaining appropriate size matching with bypassed conduits during neo-artery formation; increase the suture retention strength; provide axial and circumferential strength to withstand arterial pressures during neo-artery formation; provide kink resistance; provide prolonged durability of the inner layer; provide a composite and/or anisotropic construction; and combinations of these.

The graft devices can further include a spine or other kink-resisting element, such as to prevent luminal narrowing, radial collapse, kinking and/or other undesired movement of the graft device (e.g. movement into an undesired geometric configuration), such as while implanting the graft device during a surgical procedure and/or at a time after implantation. The spine can be placed inside the biodegradable inner layer, between the inner layer and the fiber matrix outer layer, between layers of the inner layer or the fiber matrix outer layer, and/or outside the fiber matrix outer layer. The spine can comprise a biodegradable material or otherwise be configured to provide a temporary support to the graft device. Alternatively or additionally, the spine can comprise one or more portions including durable or otherwise non-biodegradable materials configured to remain intact for long periods of time when implanted, such as at least 6 months or at least 1 year.

Also provided herein are systems and methods for producing a graft device comprising a biodegradable inner layer and a surrounding fiber matrix outer layer. Systems can include an electrospinning device and/or other fiber or fiber matrix delivering assembly. In embodiments where the graft device comprises a spine or other kink-resisting element, the spine can comprise a component that is applied, placed and/or inserted, such as by the fiber matrix delivery assembly (e.g. automatically or semi-automatically) or with a placement or insertion tool (e.g. manually).

Devices described herein can include an electrospun fiber matrix such as those disclosed in applicant's co-pending International Patent Application Serial Number PCT/US2014/065839, filed Nov. 14, 2014, the contents of which is incorporated herein by reference in its entirety. This application is directed to graft devices, as well as systems, tools and methods for producing graft devices, such as those disclosed in one or more of applicant's co-pending applications: U.S. patent application Ser. No. 13/515,996, filed Jun. 14, 2012; U.S. patent application Ser. No. 13/811,206, filed Jan. 18, 2013; U.S. patent application Ser. No. 13/979,243, filed Jul. 11, 2013; U.S. patent application Ser. No. 13/984,249, filed Aug. 7, 2013; U.S. patent application Ser. No. 14/354,025, filed Apr. 24, 2014; U.S. patent application Ser. No. 14/378,263, filed Aug. 12, 2014; and U.S. Provisional application Ser. No. 13/502,759, filed Apr. 19, 2012; the contents of each of which are incorporated herein by reference in their entirety.

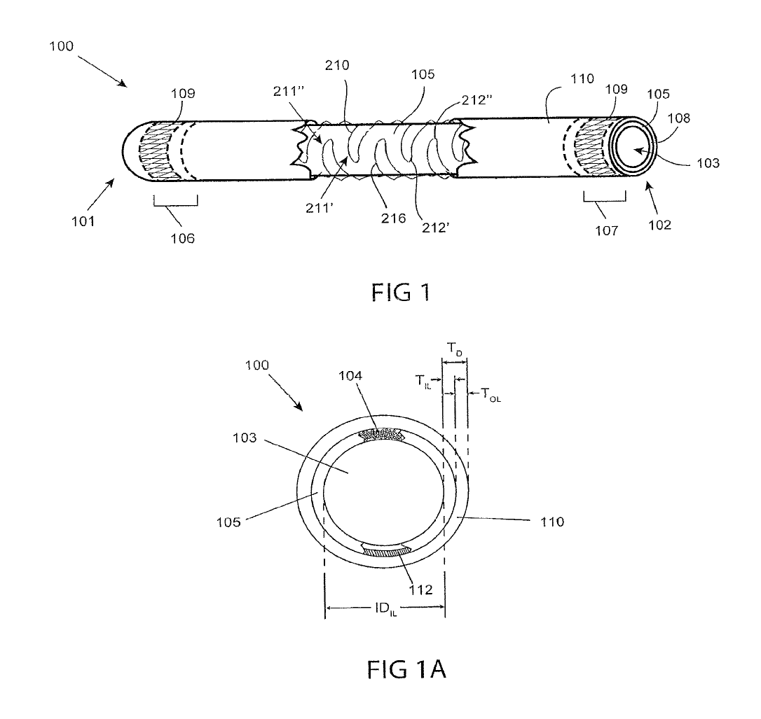

Referring now to FIG. 1, a side, partial cut-away view of an example graft device is illustrated. Graft device 100 typically includes a biodegradable inner layer 105, and an outer layer, fiber matrix 110. Inner layer 105 is circumferentially surrounded by fiber matrix 110 along the length of graft device 100. Graft device 100 includes a first end 101 and a second end 102, and is preferably configured to be placed between a first body location and a second body location of a patient. Graft device 100 includes lumen 103 from first end 101 to second end 102, such as to carry blood or other fluid when graft device 100 is connected between two body locations, such as between two blood vessels in an arterial bypass procedure. Lumen 103 can comprise a diameter between 2.0 mm and 10.0 mm, such as between 2.0 mm and 5.0 mm. In some embodiments, graft device 100 further includes spine 210 as shown. Fiber matrix 110 and/or spine 210 can also be biodegradable.

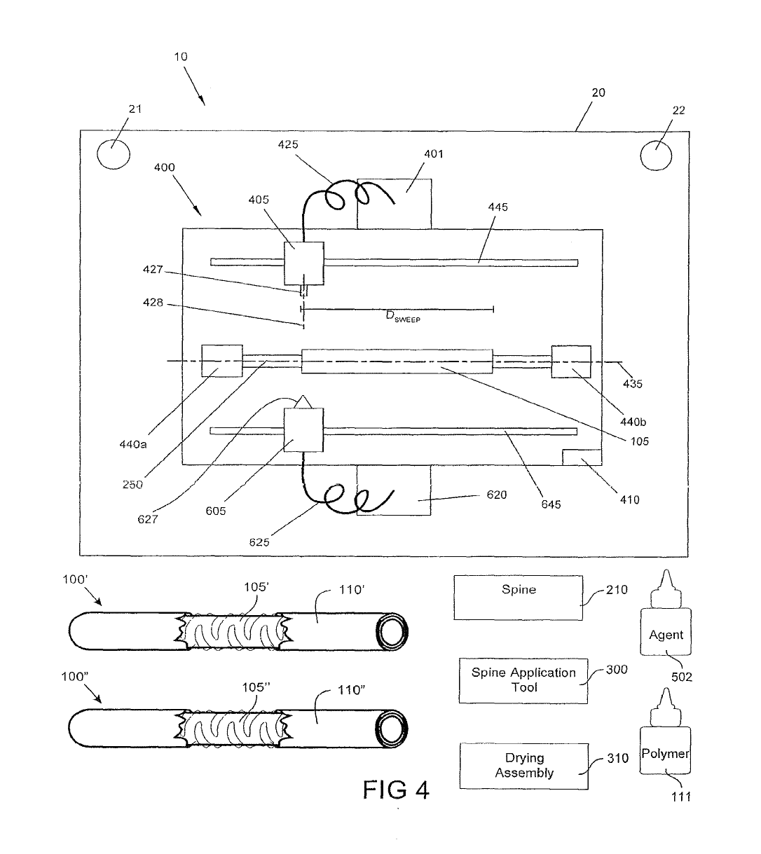

Inner layer 105 can be created with one or more devices, such as are described herebelow in reference system 10 of FIG. 4. In some embodiments, inner layer 105 is produced using a process selected from the group consisting of: particle-leaching (e.g. salt, wax and/or sugar particle leaching) following controlled dipping of a cylindrical rod into a bath of a solution containing undissolved particles of controlled size followed by dissolution of the particles to leave interconnected pores (e.g. via a freeze-drying step); particle-leaching (e.g. salt, wax and/or sugar particle leaching) following casting into a tubular mold of a solution containing undissolved particles of controlled size followed by dissolution of the particles to leave interconnected pores (e.g. via a freeze-drying step); thermally-induced separation of a solution following casting into a tubular mold followed by freeze-drying; freeze-drying of synthetic and/or biological-based hydrogels cast into a tubular mold or dipped in a bath; freeze-drying of flat sheets of de-cellularized tissues rolled onto a cylindrical template; freeze-drying of de-cellularized tubular tissues; rolling of flat sheets of synthetic meshes of material around a cylindrical template; thermoplastic extrusion of tubular constructs followed by laser excimer micro and/or macro porosity creation to form a tubular mesh structure or a porous tubular structure; sintering of thermoplastic polymer particles; wire-network molding; synthesis of a polymer with high internal phase emulsions; and combinations of these. In some embodiments, inner layer 105 is created with a device selected from the group consisting of: electrospinning device; melt-spinning device; melt-electrospinning device; 3D printer; micro-3D printer fused deposition modeling device; selective laser sintering device; laser excimer microdrilling device; sprayer; weaver; braider; knitter; dipping machine; casting machine; and combinations of these.

Inner layer 105 can comprise a varying circumferential shape (e.g. a varying diameter of its outer surface), and fiber matrix 110 and/or spine 210 can be constructed and arranged to conform to the varying circumferential shape of inner layer 105. Inner layer 105 can comprise one or more biodegradable or non-biodegradable materials. In some embodiments, inner layer 105 comprises a biodegradable polyester, such as poly(glycerol sebacate) (PGS). Alternatively or additionally, inner layer 105 can comprise other biodegradable and/or non-biodegradable materials. In some embodiments, inner layer 105 comprises a material similar to the biodegradable and/or biodegradable materials as listed for fabrication of fiber matrix 110 herebelow.

Fiber matrix 110 can be constructed and arranged as described herebelow. In some embodiments, fiber matrix 110 is created using system 10 of FIG. 4. In some embodiments, fiber matrix is constructed and arranged as described in applicant's co-pending International Patent Application Serial Number PCT/US2014/065839, filed Nov. 14, 2014, the contents of which are incorporated herein by reference in their entirety.

The thickness, compliance and biodegradation rate of inner layer 105, fiber matrix 110 and/or spine 210 can be chosen to provide an implanted support structure for a sufficient time period to allow creation of one or more new blood vessels, as has been described hereabove. In some embodiments, at least 10% of graft device 100 (e.g. at least 10% of the mass, weight, or volume of graft device 100) remains after 90 days of implantation, such as when at least 50% of graft device 100 remains after 90 days of implantation. In some embodiments, at least 10% of graft device 100 remains after 180 days of implantation, such as when at least 50% of graft device 100 remains after 180 days of implantation. In some embodiments, inner layer 105, fiber matrix 110 and/or spine 210 comprise a first material that biodegrades at a first rate, and a second material that biodegrades at a second, different rate. In some embodiments, the first material comprises a material with higher molecular weights and/or higher degrees of cross-linking than the second material, such that the first material biodegrades slower than the second material.

In some embodiments, both inner layer 105 and fiber matrix 110 biodegrade, but fiber matrix 110 biodegrades at a slower rate than inner layer 105, such as to provide sustained radial support to inner layer 105 and any remodeling tissue structures contained within fiber matrix 110 to maintain the geometric or mechanical integrity of the tissue structures over time while exposed to arterial pressures. In some embodiments, inner layer 105 can be constructed and arranged to remodel more rapidly into the functional components of blood vessels (e.g. endothelium formation, anti-thrombogenicity and medial tissue development and/or vasoactivity), while fiber matrix 110 remodels more slowly into a structural component and provides sustained support for the blood vessel (e.g. to support arterial pressure) during its remodeling. Alternatively or additionally, graft device 100 can include spine 210 to provide the necessary radial support. In some embodiments, graft device 100 includes a spine 210 that biodegrades at a much slower rate than a surrounding fiber matrix 110. In some embodiments, fiber matrix 110 comprises a tubular-net structure that surrounds inner layer 105. In some embodiments, inner layer 105 and fiber matrix 110 comprise a multiple layer (e.g. 3 or more layers) concentric structure comprising a progressively increasing continuum of biodegradation rates (e.g. with the outermost layers biodegrading at the slowest rate, or vice versa). Alternatively, the multiple layer construction can include a middle layer with a slower biodegradation rate than its two surrounding layers. In some embodiments, inner layer 105, fiber matrix 110 and/or spine 210 comprise one or more materials that exhibit surface erosion properties to a greater degree than bulk erosion properties (e.g. biodegradation is driven by surface erosion).

Referring additionally to FIG. 1A, an end view of an example graft device 100 is shown. Graft device 100 comprises a thickness T_{D}. Inner layer 105 comprises a thickness T_{IL}, and the outer layer, fiber matrix 110 comprises T_{OL}. In some embodiments, graft device 100 thickness T_{D} is related (e.g. proportional) to the inner diameter of inner layer 105, ID_{IL}, which is the same as the inner diameter of graft device 100. In some embodiments, 1/3 T_{D}<T_{IL}<1/2 T_{D}. In some embodiments, 300 μm<T_{D}<800 μm, and/or 100 μm<T_{IL}<300 μm, 200 μm <T_{OL}<500 μm. In some embodiments, layer 105 comprises a portion (e.g. a portion proximate the outer surface of inner layer 105) with minimal or no porosity (hereinafter “minimally porous” or “minimal porosity”). In these embodiments, T_{IL} can be as thick as 600 μm, of which up to 510 μm (85% of the thickness) can be constructed and arranged as an internal compressible compliance chamber, as described hereabove.

In some embodiments, inner layer 105, fiber matrix 110 and/or spine 210 comprise one or more pores, such as pores 104 shown in inner layer 105 in FIG. 1A. In some embodiments, pores 104 are positioned within an inner and/or outer layer of inner layer 105 and/or fiber matrix 110 (e.g. within a full or partial circumferential layer or sub-layer of inner layer 105 and/or fiber matrix 110). In some embodiments, pores 104 comprise pores with diameters ranging from 10 μm to 100 μm, such as between 20 μm and 30 μm. Pore 104 size distribution can vary within these ranges continuously or discretely within the radial direction of the graft device 100 wall. In some embodiments, a first set of pores 104 are positioned in proximity of the abluminal wall (i.e. outer wall) of the inner layer 105, and a second set of pores 104 are positioned in proximity of the inner wall of inner layer 105. In these embodiments, the first set of pores 104 can be of relatively larger or smaller diameter than the second set of pores 104. Pores 104 can be interconnected, such as when at least 50% of pores 104 arc interconnected. Pores 104 interconnectivity can vary continuously or discretely within the radial direction of graft device 100. For example, a first set of pores 104 in proximity to the inner wall of inner layer 105 can be more interconnected (e.g. comprising an interconnectivity of at least 80%) than a second set of pores 104 in proximity of the outer wall of inner layer 105 (e.g. comprising an interconnectivity of at least 50%), or vice-versa. In some embodiments, inner layer 105 comprises material proximate the outer wall of inner layer 105 with minimal porosity, such as an outer layer portion of inner layer 105 filled with one or more inert gasses serving as an internal compliance chamber (e.g. a foam outer layer portion of inner layer 105), such as compressible layer 112 shown in FIG. 1A. This foam or other compliant layer of inner layer 105 can be made using a multi-phase polymer solution instrument in which gas bubbles of defined size are added to the instrument prior to polymerization, solidification and/or curing by chemical agents or physical blowing. The multiphase solution can be added to a separate layer of inner layer 105 by techniques such as dipping (including rotational dipping), casting, spraying, brushing, and combinations of these. Compressible layer 112 can comprise a full or partial circumferential portion of an outer layer of inner layer 105. In some embodiments, compressible layer 112 comprises one or more layers of fiber matrix 110. Compressible layer 112 can be constructed and arranged to allow lumen 103 of graft device 100 to exhibit compliance even if one or more other portions of graft device 100 (e.g. fiber matrix 110) are relatively rigid. Inner layer 105 can comprise one or more layers (e.g. a sub-layer of inner layer 105) with a porosity configured to encourage host cell migration into inner layer 105, such as rapid cell infiltration, migration, proliferation, differentiation, or fusion to support graft remodeling that leads to a strong, compliant neo-artery.

In some embodiments, lumen 103 and/or fiber matrix 110 comprises a surface with a relatively uniform diameter along the length of graft device 100. In some embodiments, lumen 103 and/or fiber matrix 110 comprise a surface with a variable diameter, such as a tapered diameter along a segment of graft device 100 such as end portion 106 or end portion 107.

Graft device 100 can be created at a manufacturing facility or in a clinical setting, such as a sterile clinical setting within an operating room. In some embodiments, graft device 100 is created by depositing inner layer 105 and/or fiber matrix 110 over a mandrel. The mandrel can comprise a relatively straight or curved geometry as described herebelow in reference to mandrel 250 of FIG. 4. In some embodiments, graft device 100 is created in a geometry customized based on the patient's anatomy, such as by using an angio-CT or angio-MRI or other imaging techniques used to model or otherwise view the patient's anatomy.

Inner layer 105, fiber matrix 110 and/or spine 210 can comprise one or more portions that have different properties (e.g. mechanical, physical and/or chemical properties) than one or more other portions of inner layer 105, fiber matrix 110 and/or spine 210, respectively. In some embodiments, inner layer 105, fiber matrix 110 and/or spine 210 comprise two or more portions that have a dissimilar property selected from the group consisting of: biodegradation rate; morphology; pore size; porosity; permeability; anisotropy; and combinations of these. For example, graft device 100 can comprise circumferential or other portions with increased longitudinal compliance to allow stretchability and kink resistance.

Graft device 100 can comprise one or more coatings, such as one or more anti-thrombogenic (i.e. thromboresistant) coatings, such as coating 108 shown positioned on the inner surface of inner layer 105. Tn some embodiments, coating 108 comprises heparin. In some embodiments, coating 108 can be positioned on one or more surfaces of any layer of graft device 100 (e.g. inner and/or outer surface of inner layer 105 and/or a surface of any layer of fiber matrix 110) and can comprise a coating constructed and arranged to biodegrade slowly and/or at a slower rate than the material onto which coating 108 is applied. Coating 108 can be applied manually or with one or more devices, such as are described herebelow in reference to system 10 of FIG. 4. In some embodiments, coating 108 is applied with a device selected from the group consisting of: electrospinning device; melt-spinning device; melt-electrospinning device; 3D printer; fused deposition modeling device; sprayer; weaver; braider; knitter; dipping machine; casting machine; and combinations of these.

In some embodiments, coating 108 comprises tissue, such as tissue harvested from an artery or vein via a surgical instrument such as a cylindrical endothelium-tome. In these embodiments, coating 108 can comprise a thickness of 20 microns to 50 microns and it can include cells from an endothelial layer. In some embodiments, coating 108, inner layer 105, fiber matrix 110 and/or spine 210 can comprise a material configured to elude a drug or other agent configured to prevent thrombus formation.

Inner layer 105, fiber matrix 110 and/or spine 210 can comprise a component constructed of a material selected from the group consisting of: microspheres; nanoparticles such as polymer-layer silicates; metal; metal alloy; ceramic; glass; a self-assembled monolayer; a biomimetic material such as a phospholipids layer with inherent anti-thrombogenic properties; and combinations of these.

Inner layer 105, fiber matrix 110 and/or spine 210 can comprise a construction selected from the group consisting of: homogenous construction; heterogeneous construction; crystalline construction; semi-crystalline construction; amorphous construction; fibrous construction; open-celled construction; closed celled construction; woven construction; interconnected pore construction such as that produced by spherical aggregation, spherical particle-leaching (e.g. salt-leaching), thermally-induced phase separation, and/or thermally-induced particle-leaching; and combinations of these.

Graft device 100, inner layer 105 and/or fiber matrix 110 can exhibit permeability to a material selected from the group consisting of: oxygen; a cellular nutrient; cells; water; blood; and combinations of these.

In some embodiments, graft device 100 is constructed and arranged to have a limited dynamic compliance. Dynamic compliance is defined herein as the cyclic circumferential strain per unit of pressure recorded in the wall of the implanted graft device 100 as a result of cyclic pulsatile pressure in the lumen 103 of the graft device 100. Such dynamic compliance depends frequently on the luminal pressure range, in this case the pressure range that occurs during an arterial pressure cycle (e.g. standard systolic and diastolic human pressure ranges such as a pressure cycling between approximately 70 mmHg and 110 mmHg). Dynamic compliance also depends on the particular “observation point” in the radial direction of the tubular construct used to measure the cyclic strain (e.g. excursion of the inner surface of inner layer 105, the abluminal surface of inner layer 105 and/or fiber matrix 110, or a point with a given radial coordinate within a graft device 100 wall). Dynamic compliance of graft device 100 also depends on the size of lumen 103, such as a lumen 103 diameter between 2.0 mm and 5.0 mm.

Graft device 100 can be constructed and arranged to prevent undesired expansions, plastic deformations, fatigue-induced crack formations, or ruptures when exposed to cyclic arterial pressures, such as by graft device 100 comprising a dynamic compliance under a threshold (i.e. a limited dynamic compliance).

All polymers exhibit some levels of viscoelasticity, which makes them prone to creep and form cracks under cyclic loading conditions. In some embodiments, fiber matrix 110 comprises one or more materials possessing relatively high elasticity and low loss modulus (i.e. low viscoelasticity). Alternatively, in some embodiments, fiber matrix 110 comprises one or more materials with a relatively high elastic modulus (i.e. high rigidity, such as when fiber matrix 110 comprises a very low dynamic compliance such as a compliance less than 2%/100 mmHg), such as an elastic modulus high enough to substantially prevent cyclic deformations of graft device 100.

It can be desirable that dynamic compliance of graft device 100, under arterial pressures from any of the aforementioned observation points, be below 20%/100 mmHg, and in some embodiments, significantly below this value such as at a value of less than or equal to 5%/100 mmHg, for at least the radial coordinate of the abluminal layer of graft device 100 (i.e. radial coordinate=ID_{IL}/2+T_{D}). As defined by the Law of Laplace, when graft device 100 comprises elastomeric materials, the larger the inner diameter of inner layer 105, the higher the cyclic wall stress, and therefore the higher the dynamic compliance during cardiac cycles when maintaining the same wall thickness (i.e. T_{IL}+T_{OL}) and mechanical properties of graft device 100.

The resultant cyclic expansion when compliances of graft device 100 exceed the aforementioned values can prevent graft device 100 from operating effectively and/or lead to mechanical failure. For example, when compliance exceeds the value of 20%/100 mmHg, fluid dynamic disturbances can arise due to compliance mismatching and/or size-mismatching between graft device 100 and its attached vessels (e.g. the aorta and/or a bypassed coronary artery). Large excursion in the wall of graft device 100 (e.g. during high pressure) can also create regular and/or irregular plastic distensions and/or aneurysm formation (e.g. as predicted using the Law of Laplace). Excessive cyclic deformation (such as those arising from dynamic compliances greater than 20%/100 mmHg, equivalent to cyclic circumferential strains greater than 8% at each cardiac cycle and over sustained life cycles) can also generate fatigue-induced cracks, which can compromise the structural integrity of the graft device 100.

In some embodiments, fiber matrix 110 and/or spine 210 are constructed and arranged to prevent or limit such undesired expansions of graft device 100 for a sustained period of time. In some embodiments, fiber matrix 110 comprises a polymer providing sufficient rigidity and/or can include particles or other elements that provide structural reinforcement to achieve the desired minimal dynamic compliance.

In some embodiments the mechanical properties of fiber matrix 110 and/or spine 210 are sufficiently strong and rigid to prevent significant deformations and/or cyclic circumferential excursions under arterial pressure of a wall of graft device 100 (e.g. a wall including the outer or inner surface of graft device 100). In these embodiments, the material properties can exhibit minimal compliance (<1%/100 mmHg).

In some embodiments, fiber matrix 110 is constructed and arranged to provide a luminal compliance less than 20%/100 mmHg or less than 5%/100 mmHg. In these embodiments, inner layer 105 can comprise a luminal compliance greater than 20%/100 mmHg; while the limited compliance of fiber matrix 110 prevents graft device 100 from having an overall compliance greater than the compliance of fiber matrix 110.

In some embodiments, inner layer 105 can comprise one or more sub-layers with a luminal compliance (alone) less than 20%/100 mmHg or less than 5%/100 mmHg. In some embodiments, inner layer 105 comprises a compressible outer sub-layer (e.g. with minimal pore interconnectivity such as a sub-layer comprising a foam structure as described hereabove), such as compressible layer 112 described hereabove. In these embodiments, inner layer 105 can be constructed and arranged to exhibit a luminal dynamic compliance (alone) less than or equal to 20%/100 mmHg. Fiber matrix 110 can limit (e.g. substantially prevent) excursion of the abluminal coordinate of layer 105. In these embodiments, compressible layer 112 can independently cause blood flowing within lumen 103 to experience a compliance between 5%/100 mmHg and 20%/100 mmHg (e.g. independent of the compliance of fiber matrix 110).

Graft device 100 can be constructed and arranged to avoid buckling, kinking or any undesired narrowing of lumen 103 (hereinafter “kinking”). In some embodiments, graft device 100 includes one or more kink-resisting elements, such as spine 210, to prevent kinking. One or more spines 210 can be positioned on, in and/or within graft device 100, such as on the inner surface of inner layer 105, within one or more sub-layers of inner layer 105, between inner layer 105 and fiber matrix 110, within one or more sub-layers of fiber matrix 110 and/or on the outer surface of fiber matrix 110. In some embodiments, graft device 100 comprises a wall thickness T_{D} that is sufficiently large to prevent kinking, such as when inner layer 105 and/or fiber matrix 110 comprise a low density construction. In some embodiments, inner layer 105 and/or fiber matrix 110 comprise a construction possessing high longitudinal distensibility and compressibility, such as high longitudinal distensibility and compressibility compared to those along the circumferential and radial directions along the majority or specific portions of graft device 100. This particular construction can be described as exhibiting a linearly elastic behavior (without plastic deformations) along the longitudinal direction of the graft within a region comprising between −30% and +30% strain (wherein the negative sign refers to compressive strains) with an elastic modulus significantly smaller (e.g. smaller than half of the other two moduli) than those along the circumferential and radial directions of the graft. In some embodiments, inner layer 105 and/or fiber matrix 110 are constructed and arranged to include circumferential rings or bands (hereinafter “rings”) of rigid materials spaced (e.g. equally spaced) along the length of graft device 100. The rings can be positioned on, in and/or within graft device 100, such as on, in and/or within inner layer 105 or fiber matrix 110. The rings can be connected by material (e.g. tubular material) that supports axial compression and extension of graft device 100, for example during bending. In some embodiments, a ribbon (e.g. a helical ribbon) is positioned on, in or within graft device 100. In some embodiments, the rings and/or ribbons are constructed and arranged as described in applicant's co-pending U.S. patent application Ser. No. 14/378,263, filed Aug. 12, 2014, the contents of which is incorporated herein by reference in its entirety. In some embodiments, graft device 100 comprises a three dimensional deposition (e.g. via 3D printing) of a matrix of spring elements (e.g. springs) aligned along the longitudinal axis of graft device 100. The spring elements can be interconnected with struts aligned relatively orthogonal to the longitudinal axis of graft device 100. Three dimensional deposition devices can be used to create various forms of kink-resisting spines, backbones or other kink-resisting structures, such as those including loops, accordion structures and/or weaves. In some embodiments, material can be removed from inner layer 105 and/or fiber matrix 110, such as via a laser (e.g. an excimer laser), to create an accordion-like or other kink-resisting structure. In some embodiments, inner layer 105 and/or fiber matrix 110 comprise a multiple layer concentric structure in which the layers have different compliances constructed and arranged to provide kink resistance, such as when one or more inner layers have less compliance than one or more outer layers. In some embodiments, inner layer 105 and/or fiber matrix 110 comprise a ribbon that is wound to form a tubular structure. The ribbons are able to slide relative to each other to provide longitudinal distensibility that correlates to kink resistance.

In some embodiments, end portions 106 and/or 107 are constructed and arranged to provide properties (e.g. tear resistance and/or resistance to undesired stretching) suitable for supporting anastomosis of end 101 and/or 102, respectively, with suture and/or an anastomotic clip. For example, for increasing suture retention properties around an annulus of a location suitable for construction of an anastomosis, the fiber size of fiber matrix 110 could be reduced creating a softer layer to penetrate with a suture needle. A bundle of smaller fibers could have a higher suture retention strength than larger fibers due to a higher crystallinity and molecular orientation resulting from the increased elongation of the fiber during deposition. In some embodiments, coating 108 can comprise a tear-resistant coating placed on the outer surface of end portion 106 and/or 107 or at another portion 106 and/or 107 location, such as to improve the suture retention properties of graft device 100. In some embodiments, a reinforcing element 109 can he positioned on, in and/or within end portion 106 and/or 107 to provide the necessary reinforcement. Reinforcing element 109 can comprise a full circumferential structure or one or more partial circumferential structures. Reinforcing element 109 can comprise a band such as a fabric band. Reinforcing element 109 can be constructed and/or arranged in a similar fashion to spine 210 described herein. Reinforcing element 109 can comprise a cross-linking element added to inner layer 105 and/or fiber matrix 110 within end portions 106 and/or 107. Reinforcing element 109 can comprise one or more layers of inner layer 105 and/or fiber matrix 110 that are simply thicker within end portions 106 and/or 107. In some embodiments, reinforcing element 109 comprises an anastomotic clip.

In some embodiments, in order to limit (e.g. substantially prevent) a significant change in mechanical properties at the end of reinforcing element 109, reinforcing element 109 extends along the majority of the length of graft device 100, such as to be positioned in the majority of end portions 106 and 107, as well as the majority of the segment of graft device 100 in between end portions 106 and 107. Alternatively, localized reinforcement can be utilized when reinforcing element 109 is constructed and arranged to provide suture retention forces while avoiding significantly changing one or more of the other mechanical properties of graft device 100. For example, reinforcing element 109 can comprise relatively short, free-ended strands or particles that improve suture retention. Reinforcing element 109 can comprise multiple embedded, unconnected particles. Each loop of suture can pass through a single particle, such that the suture applies force to graft device 100 over an increased area (e.g. area of the particle) causing a net reduction in stress applied by the suture.

Fiber matrix 110 and/or inner layer 105 can comprise one or more materials, such as one or more similar or dissimilar polymers as described in detail herebelow. Fiber matrix 110 and/or inner layer 105 can comprise at least one polymer such as a polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations of these. The polymer can he applied in combination with a solvent where the solvent is selected from the group consisting of: hexafluoroisopropanol (HFIP); acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations of these. Fiber matrix 110 and/or inner layer 105 can comprise a thermoplastic co-polymer including two or more materials, such as a first material and a harder second material. In some embodiments, the softer material comprises segments including polydimethylsiloxane and polyhexamethylene oxide, and the harder material comprises segments including aromatic methylene diphenyl isocyanate. In some embodiments, fiber matrix 110 comprises relatively equal amounts of the softer and harder materials. In some embodiments, fiber matrix 110 comprises Elast-Eon™ material manufactured by Aortech Biomaterials of Scoresby, Australia, such as model number E2-852 with a durometer of 55D.

Fiber matrix 110 and/or inner layer 105 can comprise a biodegradable material or otherwise be configured such that the support to the graft device changes over time after implantation. Numerous biodegradable polymers can be used such as: polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations of these. Dunn et al. (U.S. Pat. No. 4,655,777) discloses a medical implant including bioabsorbable fibers that reinforce a bioabsorbable polymer matrix. Alternatively or additionally, fiber matrix 110 and/or inner layer 105 can comprise one or more portions including durable or otherwise non-biodegradable materials configured to remain intact for long periods of time when implanted, such as at least 6 months or at least 1 year.

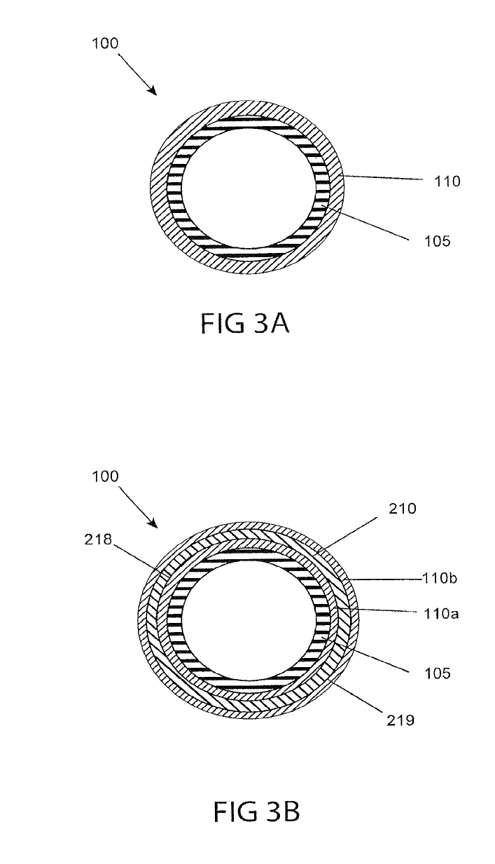

Fiber matrix 110 can comprise one or more layers, such as a fiber matrix 110 with one or more layers collectively comprising an overall thickness between 100 μm and 1000 μm, such as a thickness between 150 μm and 400 μm, between 220 μm and 280 μm, or approximately 250 μm. In some embodiments, fiber matrix 110 comprises an inner layer and an outer layer, such as an inner and outer layer with a spine 210 positioned therebetween, as described in reference to FIG. 3B herebelow. Fiber matrix 110 can comprise a matrix of fibers with an average diameter (hereinafter “diameter”) of at least 5 μm, such as a diameter between 6 μm and 15 μm, such as a matrix of fibers with an average diameter of approximately 7.8 μm or approximately 8.6 μm. Fiber matrix 110 can comprise an average porosity (hereinafter “porosity”) of between 40% and 80%, such as a fiber matrix with an average porosity of 50.4% or 46.9%. The porosity of fiber matrix 110 can be selected to control infiltration of materials into fiber matrix 110 and/or to control the rate of transmural cellular infiltration within the fiber matrix 110. In some embodiments, fiber matrix 110 comprises an average compliance (hereinafter “compliance”) between approximately 0.2×10⁻⁴/mmHg and 3.0×10⁻⁴/mmHg when measured in arterial pressure ranges. In some embodiments, fiber matrix 110 comprises an average circumferential elastic modulus (hereinafter “elastic modulus”) between 10 MPa and 18 MPa.

In some embodiments, fiber matrix 110 and/or inner layer 105 is produced by a fiber matrix delivery assembly such as an electrospinning device that converts a polymer solution into fibers applied to inner layer 105 and/or a mandrel, such as is described herebelow in reference to system 10 and electrospinning device 400 of FIG. 4. The polymer solution can comprise one or more polymers dissolved in a solvent such as hexafluoroisopropanol (HFIP). In some embodiments, at least a portion of fiber matrix 110 and/or inner layer 105 is applied with a device selected from the group consisting of: an electrospinning device; a melt-spinning device; a melt-electrospinning device; a misting assembly; a sprayer; an electrosprayer; a three-dimensional printer; and combinations of these.

Fiber matrix 110 and/or inner layer 105 can comprise one or more relatively durable (i.e. non-biodegradable) materials and/or one or more biodegradable materials. In some embodiments, fiber matrix 110 and/or inner layer 105 comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); polypropylene fumarates); a polyanhydride; a tyrosine-derived polycarbonate; a polyorthoester; a biodegradable polyurethane; a polyphosphazene; and combinations of these. Fiber matrix 110 can comprise one or more drugs or other agents, such as one or more agents constructed and arranged to be released over time.

As described hereabove, graft device 100 can further include one or more kink-resisting elements, such as spine 210. Spine 210 can be constructed and arranged to prevent graft device 100 from undergoing undesired motion such as kinking or other narrowing, such as narrowing caused during an implantation procedure and/or while under stresses endured during its functional lifespan. In some embodiments, spine 210 surrounds inner layer 105, positioned between inner layer 105 and fiber matrix 110. In these embodiments, spine 210 can comprise a diameter approximating the outer diameter (OD) of inner layer 105. In some embodiments, spine 210, in whole or in part, can be positioned between one or more layers of fiber matrix 110, such as is shown in FIG. 3B and described herebelow. In some embodiments, spine 210, in whole or in part, can surround the outer surface of fiber matrix 110. In some embodiments, spine 210 is positioned within inner layer 105. In some embodiments, multiple spines 210 can be included, each contacting the outer surface of inner layer 105, surrounding the outer surface of fiber matrix 110, and/or positioned between two or more layers of fiber matrix 110.

Fiber matrix 110 and/or spine 210 can be constructed and arranged to provide one or more functions selected from the group consisting of: minimizing undesirable conditions, such as buckling, kinking, inner layer 105 deformation, luminal deformation, stasis, flows characterized by significant secondary components of velocity vectors such as vortical, recirculating or turbulent flows, luminal collapse, and/or thrombus formation; preserving laminar flow such as preserving laminar flow with minimal secondary components of velocity, such as blood flow through graft device 100, blood flow proximal to graft device 100 and/or blood flow distal to graft device 100; preventing bending and/or allowing proper bending of graft device 100, such as bending that occurs during and/or after the implantation procedure; preventing accumulation of debris; preventing stress concentration on the tubular wall; maintaining a defined geometry in inner layer 105; preventing axial rotation about the length of inner layer 105; and combinations of these. Spine 210 and fiber matrix 110 can comprise similar elastic moduli, such as to avoid dislocations and/or separations between the two components over time, such as when graft device 100 undergoes cyclic motion and/or strain.

Spine 210 can be applied around inner layer 105 prior to, during and/or after application of fiber matrix 110 to graft device 100. For example, spine 210 can be applied prior to application of fiber matrix 110, such as when spine 210 is positioned between inner layer 105 and the inner surface of fiber matrix 110. Spine 210 can be applied during application of fiber matrix 110, such as when spine 210 is positioned between one or more layers of fiber matrix 110, as shown in FIG. 3B. Spine 210 can be applied after application of fiber matrix 110, such as when spine 210 is positioned outside of fiber matrix 110. Spine 210 can be applied about inner layer 105 and/or at least a layer of fiber matrix 110 with one or more tools, such as tool 300 described herebelow in reference to FIG. 4.

Spine 210 can include one or more portions that are resiliently biased, such as a resilient bias configured to provide a radial outward force at locations proximate ends 101 and/or 102, such as to provide a radial outward force to support or enhance the creation of an anastomosis during a cardiovascular bypass procedure. Tn some embodiments, spine 210 includes one or more portions that are malleable.

Spine 210 can include multiple curved projections 211′ and 211″, collectively 211. Projections 211′ each include a tip portion 212′ and projections 211″ each include a tip portion 212″ (collectively, tip portions 212). Tip portions 212 can be arranged in the overlapping arrangement shown in FIG. 1. Projections 211′ and 211″ can comprise a first and second support portion, respectively, that are arranged such that at least one rotates relative to the other to create an opening to receive inner layer 105. In some embodiments, each tip portion 212 can comprise a diameter between 0.020 inches and 0.064 inches, such as a diameter approximating 0.042 inches. Projections 211 can each comprise a loop of a filament (e.g. a loop of a continuous filament), and projections 211′ and 211″ can be arranged in an interdigitating arrangement such as the alternating, interdigitating arrangement shown in FIG. 1. In some embodiments, the interdigitating projections 211′ and 211″ can overlap (e.g. spine 210 covers more than 360° of inner layer 105). In some embodiments, projections 211′ and 211″ are arranged with an overlap of at least 1.0 mm, at least 1.1 mm or at least 1.4 mm. In some embodiments, spine 210 is constructed and arranged as described in applicant's co-pending International Patent Application Serial Number PCT/US2014/056371, filed Sep. 20, 2014, the contents of which is incorporated herein by reference in its entirety.

Spine 210 can comprise at least three projections 211, such as at least six projections 211. In some embodiments, spine 210 includes at least two projections 211 for every 15 mm of length of spine 210, such as at least two projections 211 for every 7.5 mm of length of spine 210, or at least two projections for every 2 mm of length of spine 210. In some embodiments, spine 210 comprises two projections 211 for each approximately 6.5 mm of length of spine 210. In some embodiments, a series of projections 211 are positioned approximately 0.125 inches from each other.

Spine 210 can comprise one or more continuous filaments 216, such as three or less continuous filaments, two or less continuous filaments, or a single continuous filament. In some embodiments, spine 210 comprises a continuous filament 216 of at least 15 inches long, or at least 30 inches long such as when spine 210 comprises a length of approximately 3.5 inches. In some embodiments, filament 216 comprises a length (e.g. a continuous length or a sum of segments with a cumulative length) of approximately 65 inches (e.g. to create a 4.0 mm diameter spine 210), or a length of approximately 75 inches (e.g. to create a 4.7 mm diameter spine 210), or a length of approximately 85 inches (e.g. to create a 5.5 mm diameter and/or 3.5 inch long spine 210). Filament 216 can comprise a relatively continuous cross section, such as an extruded or molded filament with a relatively continuous cross section. Spine 210 can comprise a filament 216 including at least a portion with a cross sectional geometry selected from the group consisting of: elliptical; circular; oval; square; rectangular; trapezoidal; parallelogram-shaped; rhomboid-shaped; T-shaped; star-shaped; spiral-shaped (e.g. a filament comprising a rolled sheet); and combinations of these. Filament 216 can comprise a cross section with a major axis between approximately 0.2 mm and 1.5 mm in length, such as a circle or oval with a major axis less than or equal to 1.5 mm, less than or equal to 0.8 mm, or less than or equal to 0.6 mm, or between 0.4 mm and 0.5 mm. Filament 216 can comprise a cross section with a major axis greater than or equal to 0.1 mm, such as a major axis greater than or equal to 0.3 mm. In some embodiments, the major axis and/or cross sectional area of filament 216 is proportionally based to the diameter of spine 210 (e.g. a larger spine 210 diameter correlates to a larger filament 216 diameter, such as when a range of different diameter spine 210′s are provided in a kit as described herebelow in reference to FIG. 4.

Filament 216 can be a single core, monofilament structure. Alternatively, filament 216 can comprise multiple filaments, such as a braided multiple filament structure. In some embodiments, filament 216 can comprise an injection molded component or a thermoset plastic component, such as when spine 210 comprises multiple projections 211 that are created at the same time as the creation of one or more filaments 216 (e.g. when filament 216 is created in a three dimensional biased shape).

Filament 216 can comprise an electrospun component, such as a component fabricated by the same electrospinning device used to create fiber matrix 110, such as when spine 210 and fiber matrix 110 comprise the same or similar materials.

Spine 210 can comprise a material with a durometer between 52D and 120R, such as between 52D and 85D, such as between 52D and 62D. In some embodiments, spine 210 comprises a material with a durometer of approximately 55D. Spine 210 can comprise one or more polymers, such as a polymer selected from the group consisting of: silicone; polyether block amide; polypropylene; nylon; polytetrafluoroethylene; polyethylene; ultra high molecular weight polyethylene; polycarbonates; polyolefins; polyurethanes; polyvinylchlorides; polyamides; polyimides; polyacrylates; polyphenolics; polystyrene; polycaprolactone; polylactic acid; polyglycolic acid; polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a biodegradable polyurethane; a polyphosphazene; and combinations of these.

Spine 210 can comprise the same material as inner layer 105 and/or fiber matrix 110. Spine 210 can comprise at least one thermoplastic co-polymer. Spine 210 can comprise two or more materials, such as a first material and a second material harder than the first material. In some embodiments, spine 210 can comprise relatively equal amounts of a harder material and a softer material. The softer material can comprise polydimethylsiloxane and a polyether-based polyurethane and the harder material can comprise aromatic methylene diphenyl isocyanate. Spine 210 can comprise one or more drugs or other agents, such as one or more agents constructed and arranged to be released over time.

In some embodiments, spine 210 comprises a metal material, such as a metal selected from the group consisting of: nickel titanium alloy; titanium alloy; titanium; stainless steel; tantalum; magnesium; cobalt-chromium alloy; gold; platinum; and combinations of these. In some embodiments, spine 210 comprises a reinforced resin, such as a resin reinforced with carbon fiber and/or Kevlar. In some embodiments, at least a portion of spine 210 is biodegradable, such as when spine 210 comprises a biodegradable material such as a biodegradable metal or biodegradable polymer. In these embodiments, fiber matrix 110 can further comprise a non-biodegradable material. In some embodiments, spine 210 does not comprise a biodegradable material.

Spine 210 can be configured to biodegrade over time such as to provide a temporary kink resistance or other function to graft device 100. In some embodiments, spine 210 can temporarily provide kink resistance to graft device 100 for a period of less than twenty-four hours. In some embodiments, spine 210 can provide kink resistance to graft device 100 for a period of less than one month. In some embodiments, spine 210 can provide kink resistance to graft device 100 for a period of less than six months. Numerous forms of metallic or non-metallic biodegradable materials can be employed. Bolz et al. (U.S. patent Ser. No. 09/339,927) discloses a bioabsorbable implant which includes a combination of metal materials that can be an alloy or a local galvanic element. Metal alloys can consist of at least a first component which forms a protecting passivation coating and a second component configured to ensure sufficient corrosion of the alloy. The first component can comprise at least one component selected from the group consisting of: magnesium, titanium, zirconium, niobium, tantalum, zinc and silicon, and the second component is at least one metal selected from the group consisting of: lithium, sodium, potassium, manganese, calcium and iron. Furst et al. (U.S. patent application Ser. No. 11/368,298) discloses an implantable device at least partially formed of a bioabsorbable metal alloy that includes a majority weight percent of magnesium and at least one metal selected from calcium, a rare earth metal, yttrium, zinc and/or zirconium. Doty (U.S. patent application Ser. No. 11/744,977) discloses a bioabsorbable magnesium reinforced polymer stent that includes magnesium or magnesium alloys. Numerous biodegradable polymers can be used such as are described hereabove.

Inner layer 105, fiber matrix 110 and/or spine 210 can comprise one or more coatings, such as coating 108 shown. Coating 108 can be positioned on an inner and/or outer surface of inner layer 105, fiber matrix 110 and/or spine 210. The one or more coatings can comprise an adhesive element or otherwise exhibit adhesive properties, such as a coating comprising a material selected from the group consisting of: fibrin gel; a starch-based compound; mussel adhesive protein; and combinations of these. The coating can be constructed and arranged to provide a function selected from the group consisting of: anti-thrombogenicity; anti-proliferation; anti-calcification; vasorelaxation; and combinations of these. A coating can comprise a dehydrated gelatin, such as a dehydrated gelatin coating configured to hydrate to cause adherence of two or more of inner layer 105, fiber matrix 110 and spine 210. A coating can comprise a hydrophilic and/or a hydrophobic coating. A coating can comprise a radiopaque coating. In some embodiments, spine 210 comprises at least a portion that is radiopaque, such as when spine 210 comprises a radiopaque material such as barium sulfate.

In some embodiments, graft device 100 is constructed and arranged to be placed in an in-vivo geometry including one or more arced portions including a radius of curvature of as low as 0.5 cm (e.g. without kinking). In some embodiments, graft device 100 is produced using system 10 and/or electrospinning device 400 of FIG. 4, as described herebelow.

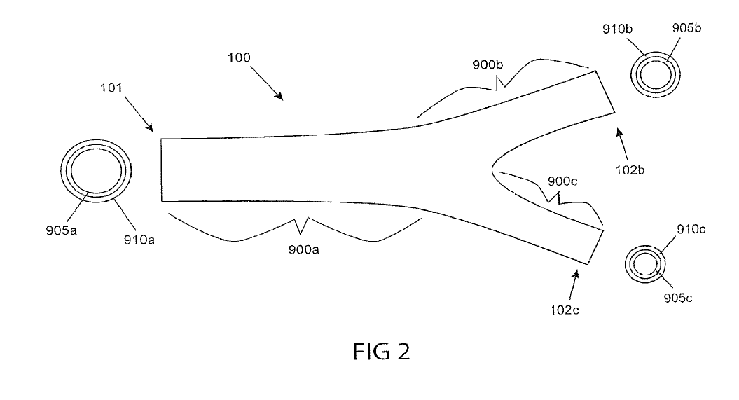

While graft device 100 of FIG. 1 is shown as a continuous, single tube construction, in some embodiments, graft devices can include multiple tubular segments such as a graft device including a bifurcation (as described herebelow in reference to FIG. 2), a trifurcation, quadrification, or other construction including one or more inflow tubes that are connected to one or more outflow tubes.

Referring now to FIG. 2, a side view of an example graft device including a bifurcation is illustrated. Graft device 100 of FIG. 2 includes a first portion 900a, a second portion 900b and a third portion 900c. First portion 900a includes inner layer 905a surrounded by an outer layer, fiber matrix 910a. Second portion 900b includes inner layer 905b surrounded by an outer layer, fiber matrix 910b. Third portion 900c includes inner layer 905c surrounded by an outer layer, fiber matrix 910c. Inner layers 905a, 905b and/or 905c can be of similar construction and arrangement to inner layer 105 of FIG. 1, as described hereabove. Fiber matrix 910a, 910b and/or 910c can be of similar construction and arrangement to fiber matrix 110 of FIG. 1, also as described hereabove. Inner layers 905a, 905b and/or 905c can comprise a porosity and/or otherwise be constructed and arranged to encourage host cell infiltration, migration, proliferation, differentiation and/or fusion followed by matrix deposition and rearrangement into the associated layer, such as rapid cell infiltration to support graft remodeling that leads to one or more strong, compliant neo-arteries. Fiber matrix 910a, 910b and/or 910c can be constructed and arranged to provide radial and other support (e.g. for a limited time period) during the creation of the one or more neo-arteries.

Graft device 100 of FIG. 2 includes an end 101 on one end of first portion 900a, an end 102b on one end of second portion 900b, and an end 102c on an end of third portion 900c. Opposite ends of portions 900a, 900b and 900c are fluidly connected, such as to create laminar flow from portion 900a to both portions 900b and 900c. In some embodiments, inner layer 905a has a greater inner diameter than the inner diameters of both inner layer 905b and 905e. In these embodiments, inner layer 905b and inner layer 905c can have similar or dissimilar inner diameters. In some embodiments, end 101 is configured to be connected to a source of body fluid, such as a source of arterial blood (e.g. the aorta). In these embodiments, ends 102b and 102e can be configured to be connected to supply blood to blood deprived tissue, such as to be each connected to an occluded artery distal to the occlusion, such as is performed in a coronary artery or peripheral artery bypass procedure. While graft device 100 of FIG. 2 includes a bifurcated geometry, constructions including multiple input or output tubes that are fluidly connected, in various geometric patterns, should be considered within the spirit and scope of the present inventive concepts.

Referring now to FIG. 3A, a sectional view of one embodiment of the graft device of FIG. 1 is illustrated, comprising an inner layer and a surrounding fiber matrix. Graft device 100 includes inner layer 105. A fiber matrix 110 has been applied about the surface of inner layer 105, such as is described in detail herebelow in reference to FIG. 4. Fiber matrix 110 can comprise one or more polymers, such as a combination of polydimethylsiloxane and polyhexamethylene oxide soft segments, and aromatic methylene diphenyl isocyanate hard segments. Fiber matrix 110 can comprise a thickness of between 220 μm and 280 μm, such as a thickness of approximately 250 μm.

Referring now to FIG. 3B, a sectional view of another embodiment of the graft device of FIG. 1 is illustrated, including a spine placed between layers of a fiber matrix. In the example depicted in FIG. 3B, spine 210 has been placed between one or more inner layers of fiber matrix 110, inner layer 110a, and one or more outer layers of fiber matrix 110, outer layer 110b. In some embodiments, spine 210 can he applied (e.g. laterally applied) to inner layer 105 after inner layer 110a has been applied to inner layer 105 by an electrospinning device or other fiber matrix delivery assembly, as described herein, such as by interrupting the delivery of fiber to inner layer 105, to apply spine 210 over the already applied inner layer 110a. In some embodiments, inner layer 110a comprises a thickness approximately one-half the thickness of outer layer 110b. In some embodiments, inner layer 110a comprises a thickness of approximately between 62 μm and 83 μm. In some embodiments, inner layer 110a comprises between 1% and 99% of the total thickness of fiber matrix 110, such as between 25% and 60% of the total thickness, or approximately 33% of the total thickness of fiber matrix 110. In some embodiments, the process time of applying inner layer 110a is between 1% and 99% of the total application time (i.e. the collective time to apply inner layer 110a and outer layer 110b ), such as between 25% and 60% of the total fiber application time, or approximately 33% of the total fiber application time.

Spine 210 comprises an inner surface 218 which contacts the outer surface of inner layer 110a. Spine 210 further comprises an outer surface 219 which contacts the inner surface of outer layer 110b. Inner surface 218, outer surface 219 and/or another surface of spine 210 can comprise a coating, such as a coating described hereabove.

Application of layers 110a and 110b can be performed as is described in detail herebelow in reference to FIG. 4. Fiber matrix layers 110a and/or 1 10b can comprise one or more polymers, such as a combination of polydimethylsiloxane and polyhexamethylene oxide soft segments, and aromatic methylene diphenyl isocyanate hard segments. Layers 110a and/or 110b can comprise a matrix of fibers with a diameter between 6 μm and 15 μm, such as a matrix of fibers with an average diameter of approximately 7.8 μm or approximately 8.6 μm. Layers 110a and/or 110b can comprise a porosity of between 40% and 80%, such as a fiber matrix with an average porosity of 50.4% or 46.9%. In some embodiments, layers 110a and/or 110b comprise a compliance between approximately 0.2×10⁻⁴/mmHg and 3.0×10⁻⁴/mmHg when measured in arterial pressure ranges. In some embodiments, fiber matrix 110 comprises an elastic modulus between 10 MPa and 18 MPa.

Referring now to FIG. 4, a schematic view of an example system for producing a graft device with an inner layer and an electrospun fiber matrix outer layer is illustrated. System 10 includes a fiber matrix delivery assembly, electrospinning device 400. System 10 is constructed and arranged to produce one or more graft devices, such as graft device 100′ or graft device 100″ shown (singly or collectively graft device 100), each including a fiber matrix, such as fiber matrix 110′ or 110″, respectively (singly or collectively fiber matrix 110), the fiber matrix 110′ or 110″ surrounding an inner layer 105′ or 105″, respectively. In some embodiments, inner layer 105′ or 105″ (singly or collectively inner layer 105) is of similar construction and arrangement as inner layer 105 of FIG. 1, described hereabove. In some embodiments, inner layer 105 is also created by electrospinning device 400. In some embodiments, inner layer 105 is created by a separate device or in a separate process, such as is described hereabove in reference to FIG. 1.

System 10 includes mandrel 250 about which inner layer 105 can be deposited. System 10 can include polymer material 111, a liquid including a mixture of one or more polymers, solvents and/or other materials used to create fiber matrix 110 and/or inner layer 105, such as are described hereabove in reference to FIG. 1. In some embodiments, system 10 comprises one or more similar or dissimilar spines 210, and graft device 100 comprises one or more of the spines 210. System 10 can include spine application tool 300, which can comprise a manual or automated (e.g. robotic) tool used to place spine 210 about inner layer 105, such as between one or more layers of fiber matrix 110 (e.g. between an inner layer with a first thickness, and an outer layer with a second thickness approximately twice as thick as the first layer's thickness). In some embodiments, graft device 100, fiber matrix 110, spine 210 and/or inner layer 105 are constructed and arranged as is described hereabove in reference to FIG. 1. In some embodiments, system 10 can include one or more tools, components, assemblies and/or otherwise be constructed and arranged as described in applicant's co-pending International Patent Application Serial Number PCT/US2014/065839, filed Nov. 14, 2014, the contents of which is incorporated herein by reference in its entirety.

As described hereabove, mandrel 250 can comprise a straight or a curved mandrel. Mandrel 250 can be radially compressible (e.g. shrinkable) or dissolvable, such as to assist in the removal from within inner layer 105 and/or fiber matrix 110 after creation of inner layer 105 and/or fiber matrix 110. Mandrel 250 can be constructed and arranged to change phase prior to removal from inner layer 105 and/or fiber matrix 110 (e.g. the material could be freeze dried, sublimated and/or melted at a low temperature to assist in the removal from inner layer 105 and/or fiber matrix 110).

Mandrel 250 can comprise a metal mandrel, such as a mandrel constructed of 304 or 316 series stainless steel. Mandrel 250 can comprise a mirror-like surface finish, such as a surface finish with an Rₐ of approximately 0.1 μm to 0.8 μm. Mandrel 250 can comprise a length of up to 45 cm, such as a length of between 30 cm and 45 cm, or between 38 cm and 40cm. In some embodiments, system 10 includes multiple mandrels 250 with multiple different geometries, such as a set of mandrels 250 with different outer diameters (e.g. diameters of 3.0 mm, 3.5 mm, 4.0 mm and/or 4.5 mm). Each end of mandrel 250 can be inserted into a rotating assembly, motors 440a and 440b, respectively, such that mandrel 250 can be rotated about axis 435 during creation of inner layer 105 and/or application of fiber matrix 110. In some embodiments, a single motor drives one end of mandrel 250, with the opposite end attached to a rotatable attachment element of electrospinning device 400.

Electrospinning device 400 can include one or more polymer delivery assemblies, and in the illustrated embodiment, device 400 includes polymer delivery assembly 405, which includes nozzle 427 including an orifice constructed and arranged to deliver inner layer 105 to mandrel 250 and/or fiber matrix 110 to inner layer 105. Nozzle 427 can be a tubular structure including nozzle central axis 428. Polymer delivery assembly 405 can be fluidly attached to polymer solution dispenser 401 via delivery tube 425. Dispenser 401 can comprise material supplied by polymer material 111 (e.g. when polymer material 111 comprises one or more polymers contained in a cartridge that is operably received by polymer solution dispenser 401). Polymer delivery assembly 405 is operably attached to a linear drive assembly 445 configured to translate polymer delivery assembly 405 in at least one direction for a linear travel distance D_{SWEEO}as shown. In some embodiments, D_{SWEEP} comprises a length of approximately 30 cm, such as a length of at least 10 cm, 20 cm, 30 cm, 35 cm or 40 cm.

In some embodiments, polymer material 111 comprises a liquid comprising two or more polymers, such as a first polymer with a first hardness, and a second polymer with a second hardness different than the first hardness. Polymer material can comprise a mixture of similar or dissimilar amounts of polyhexamethylene oxide soft segments, and aromatic methylene diphenyl isocyanate hard segments. Polymer material 111 can further comprise one or more solvents, such as HFIP (e.g. HFIP with a 99.97% minimum purity). Polymer material 111 can comprise one or more polymers in a concentrated solution fully or at least partially solubilized within a solvent and comprise a polymer weight to solvent volume ratio between 20% and 35%, a typical concentration is between 24% and 26% (more specifically between 24.5% and 25.5%). Polymer material 111 can comprise one or more materials with a molecular weight average (M_{w}) between 80,000 and 150,000 (PDI−M_{w}/Mₙ=2.1−3.5). Polymer material 111 can comprise a polymer solution with a viscosity between 2000 cP and 2400 cP (measured at 25° C. and with shear rate=20 s⁻¹). Polymer material 111 can comprise a polymer solution with a conductivity between 0.4 μS/cm and 1.7 μS/cm (measured at a temperature between 20° C. and 22° C.). Polymer material 111 can comprise a polymer solution with a surface tension between 21.5 mN/tn and 23.0 mN/m (measured at 25° C.).

In some embodiments, system 10 is constructed and arranged to produce a fiber matrix 110 with a thickness (absent of any spine 210) of between approximately 220 μm and 280 μm. In some embodiments, system 10 is constructed and arranged to produce an inner layer 105 with a thickness of between approximately 100 μm and 300 μm. Fiber matrix 110 and/or inner layer 105 can comprise a matrix of fibers with a diameter between 6 μm and 15 μm, such as a matrix of fibers with an average diameter of approximately 7.8 μm or approximately 8.6 μm. Fiber matrix 110 can comprise a porosity of between 40% and 80%, such as a fiber matrix 110 with an average porosity of 50.4% or 46.9%. Inner layer 105 can comprise a porosity of between 50% and 90%, such as an inner layer 105 with an average porosity of 70% or 85%. In some embodiments, fiber matrix 110 comprises a compliance between approximately 0.2×10⁻⁴/mmHg and 3.0×10⁻⁴/mmHg when measured in normal or moderately elevated arterial pressure ranges. In some embodiments, fiber matrix 110 comprises an elastic modulus between 10 MPa and 18 MPa. In some embodiments inner layer 105 comprises a compliance between 0.5×10⁻⁴/mmHg and 10.0×10⁻⁴/mmHg and/or elastic modulus comprised between 100 kPa and 2 MPa.

Polymer delivery assembly 405 can be configured to deliver polymer material 111 to nozzle 427 at a flow rate of between 10 ml/hr and 25 ml/hr, such as at a flow rate of approximately 15 ml/hr or 20 ml/hr.

As described above, in some embodiments, system 10 is constructed and arranged to produce a graft device 100 including a spine 210. Spine 210 can comprise multiple spines 210 with different inner diameters (IDs), such as multiple spines with IDs of approximately 3.0 mm, 3.5 mm, 4.0 mm, 4.7 mm and/or 5.5 mm. Spine 210 can comprise a filament with a diameter of approximately 0.4 mm (e.g. for a spine with an ID between 3.0 mm and 4.7 mm). Spine 210 can comprise a filament with a diameter of approximately 0.5 mm (e.g. for a spine with an ID between 4.8 mm and 5.5 mm). Spine 210 can comprise a series of inter-digitating fingers spaced approximately 0.125 inches from each other so that the recurring unit of spine including one left finger and one right finger occurs every 0.25 inches. This recurring feature length can have a range comprised between 0.125 inches and 0.375 inches. The fingers can overlap in a symmetric or asymmetric pattern, such as an overlap of opposing fingers between 2.5 mm and 1.0 mm around the circumferential perimeter of spine 210. Spine 210 can be heat treated to achieve a resilient bias. Spine 210 can be surface-treated (e.g. with dimethylformamide) to increase the surface roughness and reduce crystallinity (e.g. to improve solvent-based adhesion with the deposited electrospun material, fiber matrix 110).

System 10 can include a drying assembly 310 constructed and arranged to remove moisture from inner layer 105, fiber matrix 110 and/or another graft device 100 component. In some embodiments, drying assembly 310 comprises gauze or other material used to manually remove fluids from inner layer 105, such as to improve adherence between fiber matrix 110 and inner layer 105.

Electrospinning device 400 can include one or more graft modification assemblies constructed and arranged to modify one or more components and/or one or more portions of graft device 100. In the illustrated embodiment, device 400 includes modification assembly 605, which includes modifying element 627. Modification assembly 605 is operably attached to a linear drive assembly 645 configured to translate modification assembly 605 in at least one direction, such as a reciprocating motion in back and forth directions spanning a distance similar to D_{SWEEP} of linear drive assembly 445. Modification assembly 605 can be operably attached to supply 620 via delivery tube 625. System 10 can include one or more graft device 100 modifying agents, such as agent 502. Agent 502 can comprise a solvent configured to perform a surface modification, such as a solvent selected from the group consisting of: dimethylformamide; hexafluoroisopropanol; tetrahydrofuran; dimethyl sulfoxide; isopropyl alcohol; ethanol; and combinations of these. In some embodiments, system 10 is constructed and arranged to perform a surface modification configured to enhance the adhesion of two or more of inner layer 105, spine 210 and fiber matrix 110. In some embodiments, system 10 is constructed and arranged to perform a surface modification to inner layer 105, fiber matrix 110 and/or spine 210 to cause a modification of the surface energy of inner layer 105, fiber matrix 110 and/or spine 210, respectively. In some embodiments, the surface of spine 210 is modified with a heated die comprising a textured or otherwise non-uniform surface. In some embodiments, electrospinning device 400 and/or another component of system 10 comprise a radiofrequency plasma glow discharge assembly constructed and arranged to perform a surface modification of spine 210, such as a process performed in the presence of a material selected from the group consisting of: hydrogen; nitrogen; ammonia; oxygen; carbon dioxide; C2F6; C2F4; C3F6; C2H4; CZHZ; CH4; and combinations of these

Supply 620 can comprise one or more of: a reservoir of one or more agents such as agent 502; a power supply such as a laser power supply; and a reservoir of compressed fluid. In some embodiments, modifying element 627 comprises a nozzle, such as a nozzle configured to deliver a fiber matrix 110 modifying agent, inner layer 105 modifying agent, spine 210 modifying agent, and/or a graft device 100 modifying agent. For clarification, any reference to a “nozzle” or “assembly”, in singular or plural form, can include one or more nozzles, such as one or more nozzles 427, or one or more assemblies, such as one or more polymer delivery assemblies 405 or one or more modification assemblies 605.

In some embodiments, modifying element 627 is configured to deliver an agent 502 comprising a wax or other protective substance to inner layer 105 prior to the application of fiber matrix 110, such as to prevent or otherwise minimize exposure of inner layer 105 to one or more solvents (e.g. HFIP) included in polymer material 111.

In some embodiments, modifying element 627 is configured to deliver a kink-resisting element, for example spine 210, such as a robotic assembly constructed and arranged to laterally deliver spine 210 about at least inner layer 105 (e.g. about inner layer 105 and an inner layer of fiber matrix 110). Alternatively or additionally, modifying element 627 can be configured to modify inner layer 105, spine 210 and/or fiber matrix 110, such as to cause graft device 100 to be kink resistant or otherwise enhance the performance of the graft device 100 produced by system 10. In these embodiments in which graft device 100 is modified, modifying element 627 can comprise a component selected from the group consisting of: a robotic device such as a robotic device configured to apply spine 210 to inner layer 105; a nozzle, such as a nozzle configured to deliver agent 502; an energy delivery element such as a laser delivery element such as a laser excimer diode or other element configured to trim one or more components of graft device 100; a fluid jet such as a water jet or air jet configured to deliver fluid (e.g. a liquid and/or a gas) during the application of fiber matrix 110 to inner layer 105; a cutting element such as a cutting element configured to trim spine 210 and/or fiber matrix 110; a mechanical abrader; and combinations of these. Modification of fiber matrix 110 or other graft device 100 component by modifying element 627 can occur during the application of fiber matrix 110 and/or after fiber matrix 110 has been applied to inner layer 105. Modification of one or more spines 210 can be performed prior to and/or after spine 210 has been applied to surround inner layer 105. In some embodiments, modifying element 627 can be used to cut or otherwise trim inner layer 105, fiber matrix 110 and/or a spine 210.

In some embodiments, modification assembly 605 of system 10 can be an additional component, separate from electrospinning device 400, such as a handheld device configured to deliver spine 210. In some embodiments, modification assembly 605 comprises a handheld laser, such as a laser device which can be hand operated by an operator. Modification assembly 605 can be used to modify graft device 100 after removal of mandrel 250 and/or removal of graft device 100 from electrospinning device 400, such as prior to and/or during an implantation procedure.

Laser or other modifications to fiber matrix 110 can cause portions of fiber matrix 110 to undergo physical changes, such as hardening, softening, melting, stiffening, creating a resilient bias, expanding, and/or contracting, and/or can also cause fiber matrix 110 to undergo chemical changes, such as forming a chemical bond with an adhesive layer between the outer surface of inner layer 105 and fiber matrix 110. In some embodiments, modifying element 627 is alternatively or additionally configured to modify inner layer 105, such that inner layer 105 comprises a kink-resisting or other performance enhancing element. Modifications to inner layer 105 can include but are not limited to a physical change to one or more portions of inner layer 105 selected from the group consisting of: drying; hardening; softening; melting; stiffening; creating a resilient bias; expanding; contracting; and combinations of these. Modifications of inner layer 105 can cause inner layer 105 to undergo chemical changes, such as forming a chemical bond with an adhesive layer between an outer surface of inner layer 105 and spine 210 and/or fiber matrix 110.

As described herein, fiber matrix 110 can include an inner layer and an outer layer, where the inner layer can include an adhesive component and/or exhibit adhesive properties. The inner layer can be delivered separate from the outer layer, for example, delivered from a separate nozzle or at a separate time during the process. Selective adhesion between the inner and outer layers can be configured to provide kink resistance. Spine 210 can be placed between the inner and outer layers of fiber matrix 110, such as is described in reference to FIG. 3B hereabove.

In some embodiments, electrospinning device 400 can be configured to deliver fiber matrix 110 and/or an adhesive layer according to set parameters configured to produce a kink-resisting element in and/or provide kink-resisting properties to graft device 100. For example, an adhesive layer can be delivered to inner layer 105 for a particular length of time, followed by delivery of a polymer solution for another particular length of time. Other typical application parameters include but are not limited to: amount of adhesive layer and/or polymer solution delivered; rate of adhesive layer and/or polymer solution delivered; nozzle 427 distance to mandrel 250 and/or inner layer 105; linear travel distance of nozzle 427 or a fiber modifying element along its respective drive assembly (for example, drive assembly 445 or 645); linear travel speed of nozzle 427 or a fiber modifying element along its respective drive assembly; compositions of the polymer solution and/or adhesive layer; concentrations of the polymer solution and/or adhesive layer; solvent compositions and/or concentrations; fiber matrix 110 inner and outer layer compositions and/or concentrations; spontaneous or sequential delivery of the polymer solution and the adhesive layer; voltage applied to the nozzle; voltage applied to the mandrel; viscosity of the polymer solution; temperature of the treatment environment; relative humidity of the treatment environment; airflow within the treatment environment; and combinations of these.

Nozzle 427 can be constructed of stainless steel, such as passivated 304 stainless steel. A volume of space surrounding nozzle 427 can be maintained free of objects or substances which can interfere with the electrospinning process, such as is described in applicant's co-pending International Patent Application Serial Number PCT/US2014/065839, filed Nov. 14, 2014, the contents of which is incorporated herein by reference in its entirety. Nozzle geometry and orientation, as well as the electrical potential voltages applied between nozzle 427 and mandrel 250 are chosen to control fiber generation, such as to create an inner layer 105 and/or fiber matrix 110 as described in reference to FIG. 1 hereabove.

Mandrel 250 is positioned in a particular spaced relationship from polymer delivery assembly 405 and/or modification assembly 605, and nozzle 427 and/or modifying element 627, respectively. As illustrated, in some embodiments, mandrel 250 is positioned above and below assemblies 605 and 405, respectively. Alternatively, mandrel 250 can be positioned either above, below, to the right and/or or to the left of, assembly 405 and/or assembly 605. The distance between mandrel 250 and the tip of nozzle 427 and/or modifying element 627 can be less than 20 cm, or less than 15 cm, such as distance of between 12.2 cm and 12.8 cm or approximately 12.5 cm. In some embodiments, multiple nozzles 427 and/or multiple modifying elements 627, for example components of similar or dissimilar configurations, can be positioned in various orientations relative to mandrel 250. In some embodiments, the distance between nozzles 427 and/or modifying elements 627 and mandrel 250 varies along the length of their respective travel along mandrel 250, such as to create a varying pattern of fiber matrix 110 along inner layer 105. In some embodiments, nozzle 427 and/or modifying element 627 distances from mandrel 250 can vary continuously during the electrospinning process and/or the distance can vary for one or more set periods of time during the process.

In some embodiments, an electrical potential is typically applied between nozzle 427 and one or both of inner layer 105 and mandrel 250. The electrical potential can draw at least one fiber from polymer delivery assembly 405 to inner layer 105. Inner layer 105 can act as the substrate for the electrospinning process, collecting the fibers that are drawn from polymer delivery assembly 405 by the electrical potential. In some embodiments, mandrel 250 and/or inner layer 105 has a lower voltage than nozzle 427 to create the desired electrical potential. For example, the voltage of mandrel 250 and/or inner layer 105 can be a negative or zero voltage while the voltage of nozzle 427 can be a positive voltage. Mandrel 250 and/or inner layer 105 can have a voltage of about −5 kV (e.g. −10 kV, −9 kV, −8 kV, −7 kV, −6 kV, −5 kV, −4.5 kV, −4 kV, −3.5 kV, −3.0 kV, −2.5 kV, −2 kV, −1.5 kV or −1 kV) and the nozzle 427 can have a voltage of about +15 kV (e.g. 2.5 kV, 5 kV, 7.5 kV, 12 kV, 13.5 kV, 15 kV, 17 kV or 20 kV). In some embodiments, the potential difference between nozzle 427 and mandrel 250 and/or inner layer 105 can be from about 5 kV to about 30 kV. This potential difference draws fibers from nozzle 427 to inner layer 105. In some embodiments, nozzle 427 is electrically charged with a potential of between +15 kV and +17 kV while mandrel 250 is at a potential of approximately −2 kV. In some embodiments, mandrel 250 is a fluid mandrel, such as the fluid mandrel described in applicant's co-pending PCT Application Serial Number PCT/US2011/066905 filed on Dec. 22, 2011, the contents of which are incorporated herein by reference in their entirety.

In some embodiments, system 10 comprises a polymer solution, such as polymer material 111. Polymer material 111 can be introduced into polymer solution dispenser 401, and then delivered to polymer delivery assembly 405 through polymer solution delivery tube 425. The electrical potential between nozzle 427 and inner layer 105 and/or mandrel 250 can draw the polymer solution through nozzle 427 of polymer delivery assembly 405. Electrostatic repulsion, caused by the fluid becoming charged from the electrical potential, counteracts the surface tension of a stream of the polymer solution at nozzle 427 of the polymer delivery assembly 405. After the stream of polymer solution is stretched to its critical point, one or more streams of polymer solution emerges from nozzle 427 of polymer delivery assembly 405, and/or at a location below polymer delivery assembly 405, and move toward the negatively charged inner layer 105. Using a volatile solvent, the solution dries substantially during transit and fiber is applied about inner layer 105 creating fiber matrix 110.

Mandrel 250 is configured to rotate about an axis, such as central axis 435 of mandrel 250, with axis 428 of nozzle 427 typically oriented orthogonal to axis 435. In some embodiments, axis 428 of nozzle 427 is horizontally offset from axis 435. The rotation around axis 435 allows fiber matrix 110 to be applied along all sides, or around the entire circumference of inner layer 105. In some embodiments, two motors 440a and 440b are used to rotate mandrel 250. Alternatively, electrospinning device 400 can include a single motor configured to rotate mandrel 250 as described hereabove. The rate of rotation of mandrel 250 can determine how the electrospun fibers are applied to one or more segments of inner layer 105. For example, for a thicker portion of fiber matrix 110, the rotation rate can be slower than when a thinner portion of fiber matrix 110 is desired. In some embodiments, mandrel 250 is rotated at a rate of between 100 rpm and 400 rpm, such as a rate of between 200 rpm and 300 rpm, between 240 rpm and 260 rpm, or approximately 250 rpm.

In addition to mandrel 250 rotating around axis 435, the polymer delivery assembly 405 can move, such as when driven by drive assembly 445 in a reciprocating or oscillating horizontal motion (to the left and right of the page). Drive assembly 445, as well as drive assembly 645 which operably attaches to modification assembly 605, can each comprise a linear drive assembly, such as a belt-driven drive assembly comprising two or more pulleys driven by one or more stepper motors. Additionally or alternatively, assemblies 405 and/or 605 can be constructed and arranged to rotate around axis 435, rotating means not shown. The length of drive assemblies 445 and/or 645 and the linear motion applied to assemblies 405 and 605, respectively, can vary based on the length of inner layer 105 to which a fiber matrix 110 is delivered and/or a fiber matrix 110 modification is applied. For example, the supported linear motion of drive assemblies 445 and/or 645 can be about 10 cm to about 50 cm, such as to cause a translation of assembly 405 and/or assembly 605 between 27 cm and 31 cm, or approximately 29 cm. Rotational speeds of mandrel 250 and translational speeds of assemblies 405 and/or 605 can be relatively constant, or can be varied during the fiber application process. In some embodiments, assembly 405 and/or 605 are translated (e.g. back and forth) at a relatively constant translation rate between 40 mm/sec and 150 mm/sec, such as to cause nozzle 427 and/or modifying element 627 to translate at a rate of between 50 mm/sec and 80 mm/sec, between 55 mm/sec and 65 mm/sec, or approximately 60 mm/sec, during the majority of its travel. In some embodiments, system 10 is constructed and arranged to rapidly change directions of translation (i.e. maximize deceleration before a direction change and/or maximize acceleration after a direction change).

Assemblies 405 and/or 605 can move along the entire length or specific portions of the length of inner layer 105. In some embodiments, fiber and/or a modification is applied to the entire length of inner layer 105 plus an additional 5 cm (to mandrel 250) on either or both ends of inner layer 105. In some embodiments, fiber(s) and/or a modification is applied to the entire length of inner layer 105 plus at least 1 cm beyond either or both ends of inner layer 105. Assemblies 405 and/or 605 can be controlled such that specific portions along the length of inner layer 105 are reinforced with a greater amount of fiber matrix 110 as compared to other or remaining portions (e.g. greater thickness of fiber matrix 110 at one or more locations). Alternatively or additionally, assemblies 405 and/or 605 can be controlled such that specific portions of the length of inner layer 105 include one or more kink-resisting elements (e.g. one or more spines 210) positioned at those one or more specific inner layer 105 portions. In addition, inner layer 105 can be rotating around axis 435 while assemblies 405 and/or 605 move, via drive assemblies 445 and/or 645, respectively, to position assemblies 405 and/or 605 at the particular portion of inner layer 105 to which fiber is applied and/or modified.

System 10 can also include a power supply, power supply 410 configured to provide the electric potentials to nozzle 427 and mandrel 250, as well as to supply power to other components of system 10 such as drive assemblies 445 and 645 and modification assembly 605. Power supply 410 can be connected, either directly or indirectly, to at least one of mandrel 250 or inner layer 105. Power can be transferred from power supply 410 to each component by, for example, one or more wires.

System 10 can include an environmental control assembly including environmental chamber 20 that surrounds electrospinning device 400. System 10 can be constructed and arranged to control the environmental conditions within chamber 20, such as to control one or more environment surrounding polymer delivery assembly 405 and/or mandrel 250 during the application of inner layer 105 to mandrel 250 and/or application of fiber matrix 110 to inner layer 105. Chamber 20 can include inlet port assembly 21 and outlet port assembly 22. Inlet port assembly 21 and/or outlet port assembly 22 can each include one or more components such as one or more components selected from the group consisting of: a fan; a source of a gas such as a dry compressed air source; a source of gas at a negative pressure; a vapor source such as a source including a buffered vapor, an alkaline vapor and/or an acidic vapor; a filter such as a HEPA filter; a dehumidifier; a humidifier; a heater; a chiller; and electrostatic discharge reducing ion generator; and combinations of these. Chamber 20 can include one or more environmental control components to monitor and/or control temperature, humidity and/or pressure within chamber 20. Chamber 20 can be constructed and arranged to provide relatively uniform ventilation about mandrel 250 (e.g. about inner layer 105, fiber matrix 110 and/or spine 210) including an ultra-dry (e.g. <2 ppm water or other liquid content) compressed gas (e.g. air) source to reduce humidity. Inlet port 21 and outlet port 22 can be oriented to purge air from the top of chamber 20 to the bottom of chamber 20 (e.g. to remove vapors of one or more solvents (e.g. HFIP) which can tend to settle at the bottom of chamber 20). Chamber 20 can be constructed and arranged to replace the internal volume of chamber 20 at least once every 3 minutes, or once every 1 minute, or once every 30 seconds. Outlet port 22 can include one or more filters (e.g. replaceable cartridge filters) which are suitable for retaining halogenated solvents or other undesired materials evacuated from chamber 20. Chamber 20 can be constructed and arranged to maintain a flow rate through chamber 20 of at least 30 L/min, such as at least 45 L/min or at least 60 L/min during an initial purge procedure. Subsequent to the initial purge procedure, a flow rate of at least 5 L/min, at least 10 L/min, at least 20 L/min or at least 30 L/min can be maintained, such as to maintain a constant humidity level (e.g. a relative humidity between 20% and 24%). Chamber 20 can be further constructed and arranged to control temperature, such as to control temperature within chamber 20 to a temperature between 15° C. and 25° C., such as between 16° C. and 20° C. with a relative humidity between 20% and 24%. In some embodiments, one or more objects or surfaces within chamber 20 are constructed of an electrically insulating material and/or do not include sharp edges or exposed electrical components. In some embodiments, one or more metal objects positioned within chamber 20 are electrically grounded.

In some embodiments, system 10 is configured to produce a graft device 100′ based on one or more component or process parameters. In these embodiments, graft device 100′ comprises inner layer 105′ and a fiber matrix 110′, either or both applied by electrospinning device 400. Inner layer 105′ and/or fiber matrix 110′ can be applied via polymer delivery assembly 405 supplied with polymer material 111 at a flow rate of approximately 15 ml/hr. Inner layer 105′ and/or fiber matrix 110′ can be applied when an electrostatic potential of approximately 17 kV is applied between nozzle 427 and mandrel 250, such as when nozzle 427 is charged to a potential of approximately +15 kV and mandrel 250 is charged to a potential of approximately −2 kV. Cumulative application time of fiber matrix 110′ can comprise an approximate time period of between 11 minutes and 40 seconds and 17 minutes and 30 seconds. The cumulative application time of fiber matrix 110′ can comprise a time period of approximately 11 minutes and 40 seconds when inner layer 105′ comprises an outer diameter of between approximately 3.4 mm and 4.2 mm, a time period of approximately 14 minutes and 0 seconds when inner layer 105′ comprises an outer diameter between approximately 4.2 mm and 5.1 mm, and/or a time period of approximately 17 minutes and 30 seconds when inner layer 105′ comprises an outer diameter between approximately 5.1 mm and 6.0 mm.

Inner layer 105′ and/or fiber matrix 110′ can comprise an average fiber size of approximately 7.8 μm, such as a population of fiber diameters with an average fiber size of approximately 7.8 μm with a standard deviation of 0.45 μm. Inner layer 105′ and/or fiber matrix 110′ can comprise an average porosity of approximately 50.4%, such as a range of porosities with an average of 50.4% and a standard deviation of 1.1%. Inner layer 105′ and/or fiber matrix 110′ can comprise a strength property selected from the group consisting of: stress measured at 5% strain comprising between 0.4 MPa and 1.1 MPa; ultimate stress of 4.5 MPa to 7.0 MPa; ultimate strain of 200% to 400%; and combinations of these. Inner layer 105′ and/or fiber matrix 110′ can comprise a compliance between approximately 0.2×10⁻⁴/mmHg and 3.0×10⁻⁴/mmHg when measured in arterial pressure ranges. Inner layer 105′ and/or fiber matrix 110′ can comprise an elastic modulus between 10 MPa and 15 MPa. Inner layer 105′ and/or fiber matrix 110′ can be constructed and arranged with a targeted suture retention strength, such as an approximate suture retention strength of between 2.0N and 4.0N with 6-0 Prolene suture and/or between 1.5N and 3.0N with 7-0 Prolene suture. In some embodiments, graft device 100′ includes a spine 210, such as a spine 210 placed between inner and outer layers of fiber matrix 110′ (e.g. placed after one-third of the total thickness of fiber matrix 110′ is applied about inner layer 105′).

In some embodiments, system 10 is configured to produce a graft device 100″ based on one or more component or process parameters. In some examples, graft device 100″ comprises inner layer 105″ and a fiber matrix 110″, either or both applied by electrospinning device 400.. Inner layer 105″ and/or fiber matrix 110″ can be applied via polymer delivery assembly 405 supplied with polymer material 111 at a flow rate of approximately 20 ml/hr. Inner layer 105″ and/or fiber matrix 110″ can be applied when an electrostatic potential of approximately 19 kV is applied between nozzle 427 and mandrel 250, such as when nozzle 427 is charged to a potential of approximately +17 kV and mandrel 250 is charged to a potential of approximately −2 kV. Cumulative application time of fiber matrix 110″ can comprise an approximate time period of between 9 minutes and 30 seconds and 13 minutes and 40 seconds. The cumulative application time of fiber matrix 110″ can comprise a time period of approximately 9 minutes and 30 seconds when inner layer 105″ comprises an outer diameter between approximately 3.4 mm and 4.2 mm; a time period of approximately 11 minutes and 30 seconds when inner layer 105″ comprises an outer diameter between approximately 4.2 mm and 5.1 mm, and/or a time period of approximately 13 minutes and 40 seconds when inner layer 105″ comprises an outer diameter between approximately 5.2 mm and 6.0 mm.

Inner layer 105″ and/or fiber matrix 110″ can comprise an average fiber size of approximately 8.6 μm, such as a population of fiber diameters with an average fiber size of approximately 8.6 μm with a standard deviation of 0.45 μm. Inner layer 105″ and/or fiber matrix 110″ can comprise an average porosity of approximately 46.9%, such as a range of porosities with an average of 46.9% and a standard deviation of 0.9%. Inner layer 105″ and/or fiber matrix 110″ can comprise a strength property selected from the group consisting of: stress at 5% strain comprising between 0.6 MPa and 1.3 MPa; ultimate stress of 5.0 MPa to 7.5 MPa; ultimate strain of 200% to 400%; and combinations of these. Inner layer 105″ and/or fiber matrix 110″ can comprise an average compliance (hereinafter “compliance”) between approximately 0.2×10⁻⁴/mmHg and 3.0×10⁻⁴/mmHg when measured in arterial pressure ranges. Inner layer 105″ and/or fiber matrix 110″ can comprise an elastic modulus between 12 MPa and 18 MPa. Inner layer 105″ and/or fiber matrix 110″ can be constructed and arranged with a targeted suture retention strength, such as an approximate suture retention strength of between 2.3N and 4.3N with 6-0 Prolene suture and/or between 2.0 N and 3.5 N with 7-0 Prolene suture. In some embodiments, graft device 100″ includes a spine 210, such as a spine 210 placed between inner and outer layers of fiber matrix 110″ (e.g. placed after one-third of the total thickness of fiber matrix 110″ is applied about inner layer 105″).

Fiber matrix 110″ of graft device 100″ can comprise more bonds between fibers than fiber matrix 110′ of graft device 100′. The increased number of bonds can result in a higher fiber matrix 110″ density which can be configured to limit cellular infiltration into graft device 100″ (e.g. to increase the graft durability in vivo). Fiber matrix 110″ can comprise fibers that are flatter (i.e. more oval versus round) and/or denser than fibers of fiber matrix 110′. Fiber matrix 110″ can have a greater resiliency than fiber matrix 110′. Inner layer 105′ and 105″ can have one or more similar differences.

In some embodiments, device 400, tool 300 and/or another component of system 10 is constructed and arranged to position a reinforcing element in an end portion of a graft device 100, such as reinforcing element 109 positioned in end portions 106 and/or 107 of graft device 100 of FIG. 1, described hereabove.

While the graft devices herein have generally been described in detail as including an electrospun inner layer 105 and/or fiber matrix 110, other fiber delivery or other material application equipment can be used. The graft devices can include one or more spines, or the inner layer 105 and/or applied fiber matrix 110 can be configured to sufficiently resist kinking without the inclusion of the spine.

While some embodiments of the systems, methods and devices have been described in reference to the environment in which they were developed, they are merely illustrative of the principles described herein. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it can be possible, or even expedient in certain circumstances, to change the order in which some steps are performed, and it is intended that the particular steps of the method or procedure claim set forth herebelow not be construed as being order-specific unless such order specificity is expressly stated in the claim.

1-143. (canceled)

144. A graft device comprising:
a first end portion, a second end portion, and a lumen therebetween;
an inner layer comprising an inner surface and an outer surface; and
an outer layer comprising a fiber matrix and circumferentially surrounding the outer surface of the inner layer,
wherein said outer layer comprises a kink-resisting structure formed by removing material from said fiber matrix,
wherein the inner layer comprises a portion permeable to cells; and
wherein the inner layer is configured to biodegrade faster than the outer layer.

145. The graft device according to claim 144, wherein the fiber matrix comprises a biodegradable material.

146. The graft device of claim 144, wherein the fiber matrix comprises a non-biodegradable material.

147. The graft device according to claim 144, wherein the fiber matrix comprises both a biodegradable material and a non-biodegradable material.

148. The graft device of claim 144, further comprising pores positioned in at least one of the inner layer or the outer layer.

149. The graft device of claim 148, wherein the pores comprise diameters ranging from 10 μm to 100 μm.

150. The graft device of claim 149, wherein the pores comprise diameters ranging from 20 μm to 30 μm.

151. The graft device of claim 148, wherein the pores comprise a first set of pores and a second set of pores, and wherein the first set of pores comprises an average diameter different than an average diameter of the second set of pores.

152. The graft device of claim 148, wherein the pores comprise interconnecting pores.

153. The graft device of claim 152, wherein an interconnectivity of the interconnecting pores varies along a radial direction of the graft device.

154. The graft device of claim 152, wherein the pores comprise a first set of pores proximate the inner surface and a second set of pores proximate the outer surface, and wherein the first set of pores comprise an interconnectivity higher than an interconnectivity of the second set of pores.

155. The graft device of claim 144, wherein the inner layer comprises a biodegradable polyester.

156. The graft device of claim 144, wherein the inner layer comprises a first material and a second material.

157. The graft device of claim 156, wherein the first material and the second material are constructed and arranged to biodegrade at different rates.

158. The graft device of claim 156, wherein the first material and the second material comprise different molecular weights.

159. The graft device of claim 156, wherein the first material and the second material comprise different degrees of cross-linking.

160. The graft device of claim 144, wherein the inner layer further comprises a non-biodegradable material.

161. The graft device of claim 144, wherein the inner layer is constructed and arranged to biodegrade primarily via surface erosion.

162. The graft device of claim 144, further comprising a coating.

163. The graft device of claim 162, wherein the coating comprises a thromboresistant coating.

164. The graft device of claim 163, wherein the thromboresistant coating comprises heparin.

165. The graft device of claim 163, wherein the thromboresistant coating comprises a coating positioned on the inner surface of the inner layer.

166. The graft device of claim 162, wherein the coating comprises an adhesive.

167. The graft device of claim 166, wherein the adhesive coating comprises a coating positioned on the outer surface of the inner layer.

168. The graft device of claim 162, wherein the coating is constructed and arranged to provide a function selected from the group consisting of: anti-thrombogenicity; anti-proliferation; anti-calcification; vasorelaxation; and combinations thereof.

169. The graft device of claim 144, wherein the inner layer further comprises one or more of: microspheres; nanoparticles; nanoparticles comprising polymer-layer silicates; metal; metal alloy; ceramic; glass; a self-assembled monolayer; biomimetic material; a phospholipids layer with inherent anti-thrombogenic properties; or combinations thereof.

170. The graft device of claim 144, wherein the inner layer comprises a construction selected from the group consisting of: homogenous construction; heterogeneous construction; crystalline construction; semi-crystalline construction; amorphous construction; fibrous construction; open-celled construction; closed celled construction; woven construction; interconnected pore construction; an interconnected pore construction produced by spherical aggregation; an interconnected pore construction produced by spherical particle-leaching; an interconnected pore construction produced by salt-leaching; an interconnected pore construction produced by thermally-induced phase separation; an interconnected pore construction produced by thermally-induced particle-leaching; and combinations thereof.

171. The graft device of claim 144, wherein said kink-resisting structure is accordion-like.

172. The graft device of claim 144, wherein said outer layer comprises a modification formed by hardening, softening, melting, expanding, or contracting a portion of said fiber matrix.

173. The graft device of claim 144, wherein said outer layer comprises a modification applied along an entire length of said inner layer.
